(19) United States
(12) Patent Application Publication
(10) Pub. No.: US 2005/0019902 A1
(43) Pub. Date: Jan. 27, 2005

(54) Miniaturized integrated nucleic acid processing and analysis device and method

(76) Inventors: Richard A. Mathies, Moraga, CA (US); Eric T. Lagally, Berkeley, CA (US); Peter C. Simpson, San Francisco, CA (US)

Correspondence Address: CHIEF INTELLECTUAL PATENT COUNSEL;AFFYMETRIX, INC., 3380 CENTRAL EXPRESSWAY, SANTA CLARA, CA, 95051, US

(21) Appl. No.: 10/918,575

(22) Filed: Aug. 14, 2004

Related U.S. Application Data
(63) Continuation of application No. 09/927,431, filed on Aug. 9, 2001.
Application No. 09/927,431 is a continuation-in-part of application No. 09/651,532, filed on Aug. 29, 2000, now Pat. No. 6,284,525.
Application No. 09/651,532 is a continuation of application No. 08/535,875, filed on Sep. 28, 1995, now Pat. No. 6,132,580.
(60) Provisional application No. 60/224,195, filed on Aug. 9, 2000.

(51) Int. Cl. C12M 1/38

(52) U.S. Cl. 435/287.2; 435/286.1; 435/303.1

(57) ABSTRACT

A miniature device has a body including one, two or more reaction chambers. The reaction chambers are constructed for one or more of the following: sample acquisition, preparation or analysis. Preferably, a sample preparation reaction includes nucleic acid extraction, amplification, nucleic acid fragmentation, labeling, extension or a transcription.

This application is a continuation of U.S. application Ser. No. 09/927,431, filed on Aug. 9, 2001, which is a continuation-in-part of U.S. application Ser. No. 09/651,532, filed on Aug. 29, 2000, which is a continuation of U.S. application Ser. No. 08/535,875, filed on Sep. 28, 1995, now U.S. Pat. No. 6,132,580. The U.S. application Ser. No. 09/927,431 also also claims priority from U.S. Provisional Application No. 60/224,195 filed on Aug. 9, 2000. The disclosure of all of the above-mentioned applications is considered part of and is incorporated by reference in the disclosure of this application.

### BACKGROUND OF THE INVENTION

The present invention relates to processing and analyzing biological materials, and in particular relates to a device for carrying out a variety of synthetic and diagnostic applications, such as PCR amplification, nucleic acid hybridization, chemical labeling, thermal cycling, nucleic acid fragmentation, transcription, or various sequence based analyses.

The relationship between structure and function of macromolecules is of fundamental importance in the understanding of biological systems. This relationship is important to understanding, for example, the functions of enzymes, structure of signaling proteins, ways in which cells communicate with each other, as well as mechanisms of cellular control and metabolic feedback.

Genetic information is critical in continuation of life processes. Life is substantially informationally based; its genetic content controls the growth and reproduction of the organism. The amino acid sequences of polypeptides, which are critical features of all living systems, are encoded by the genetic material of the cell. Further, the properties of these polypeptides, e.g., as enzymes, functional proteins, and structural proteins, are determined by the sequence of amino acids that make them up. As structure and function are integrally related, many biological functions may be explained by elucidating the underlying structural features that provide those functions, and these structures are determined by the underlying genetic information in the form of polynucleotide sequences. In addition to encoding polypeptides, polynucleotide sequences can also be specifically involved in, for example, the control and regulation of gene expression.

The study of this genetic information has proved to be of great value in providing a better understanding of life processes, as well as diagnosing and treating a large number of disorders. In particular, disorders which are caused by mutations, deletions or repetitions in specific portions of the genome, may be potentialy diagnosed and/or treated using genetic techniques. Similarly, disorders caused by external agents may be diagnosed by detecting the presence of genetic material that is unique to the external agent, e.g., by detecting DNA of a specific bacteria or virus.

Current genetic methods are generally capable of identifying these genetic sequences by relying on a multiplicity of distinct processes. These processes generally draw from a large number of distinct disciplines, including chemistry, molecular biology, medicine and others.

A large number of diagnostic and synthetic chemical reactions require precise monitoring and control of reaction parameters for small volumes of samples. For example, in nucleic acid based diagnostic applications, it is generally desirable to maintain optimal temperature controls for a number of specific operations in the overall process. In particular, PCR amplification requires repeated cycling through a number of specific temperatures to carry out the melting, annealing, and ligation steps that are part of the process. By reducing reaction volumes, the amount of time required for thermal cycling may also be reduced, thereby accelerating the amplification process. Further, this reduction in volume also results in a reduction of the amounts of reagents and sample used, thereby decreasing costs and facilitating analyses of small amounts of material. Similarly, in hybridization applications, precise temperature controls are used to obtain optimal hybridization conditions. Finally, a number of other pre- and posthybridization treatments also favor precise temperature control, such as fragmentation, transcription, chain extension for sequencing, labeling, ligation reactions, and the like.

Various miniature and integrated reaction vessels for carrying out a variety of chemical reactions, including nucleic acid manipulation have been described. For example, PCT publication WO 94/05414 reports an integrated micro-PCR apparatus fabricated from thin silicon wafers, for collection and amplification of nucleic acids from a specimen. U.S. Pat. No. 5,304,487 to Wilding, et al., and U.S. Pat. No. 5,296,375 to Kricka, et al. discuss micromachined chambers and flow channels for use in collection and analysis of cell samples.

However, there is still a need to integrate various processes for sample preparation, processing and analysis into a single device or a small number of devices that can handle small samples, are highly accurate, and are relatively inexpensive.

### SUMMARY OF THE INVENTION

The present invention relates to a system and method for processing and analyzing biological materials.

According to another aspect, a miniature device has a body including one, two or more reaction chambers. The reaction chamber may be constructed for one or more of the following: sample acquisition, preparation or analysis. Preferably, a sample preparation reaction chamber may include a nucleic acid extraction chamber, an amplification chamber, a nucleic acid fragmentation chamber, a labeling chamber, an extension reaction chamber, or a transcription reaction chamber. Preferably, the analysis chamber (or in general analytical device) may include an oligonucleotide probe array, an electrophoresis device, or another sequencing device. The electrophoresis device may be a microcapillary electrophoresis device.

Preferably, the analysis chamber may include an oligonucleotide probe array located the wall of the chamber (i.e., a wall forming an integral part of the device body) or located on a substrate removable from the device. This substrate may be attachable to and may form a removable wall of the analysis chamber. The substrate may include a plurality of positionally distinct oligonucleotide probes coupled to the surface of the substrate. The substrate may be transparent. The analysis chamber may be co-operatively arranged to have said substrate readable by a fluorescent microscope. The analysis chamber may be co-operatively arranged to have said substrate readable by a confocal or pseudoconfocal fluorescent microscope.

Alternatively, the analysis chamber includes a microcapillary electrophoresis device (which actually is not disposed in one chamber but includes several capillaries) as described in U.S. Pat. No. 6,132,580 or U.S. Pat. No. 6,168,948. Alternatively, the analytical device includes one or several systems described in PCT publication WO 00/09757, which is incorporated by reference in its entirety. The miniature device described below has one of several reaction chambers arranged to include a polymer supply station, a polymer alignment station, a first interaction station, and a second interaction station (all described in the PCT publication WO 00/09757) all being fabricated on one substrate. A processed sample is delivered via microfluidic channels to these analysis stations for sample analysis and sequencing. Alternatively, the analytical device described in PCT publication WO 00/09757 may be external to the present miniature device.

Preferably, the body of the miniature device includes at least first and second planar members, wherein the first planar member has a first surface and a well disposed in the first surface, and the second planar member has a second surface being mated to the first surface whereby the well forms the cavity.

Preferably, an acquisition, preparation or analysis chamber includes a resistive heater and a temperature sensor deposited within its cavity or on the wall of the chamber. The heater is electrically connected to a power source for applying controlled amounts of power to the heater controlled by a controller. The power source may deliver an AC voltage across the resistive heater. The resistive heater may include a chromium film connected by electrical connections, including two gold leads overlaying the chromium film, to the power source. The chromium film may be between about 250 Å and about 4,000 Å thick and the chromium layer may be between about 200 Å and 300 Å thick. Furthermore, an acquisition, preparation or analysis chamber may include a thermoelectric cooler.

Preferably, the temperature sensor may be is deposited on the second surface, wherein when the second surface is mated with the first surface, the temperature sensor on the second surface is positioned within the cavity whereby a temperature at the temperature sensor is substantially the same as a temperature of the cavity. The temperature sensor may include a thermocouple having a sensing junction and a reference junction. The sensing junction may be positioned in or adjacent to the cavity. The reference junction is usually positioned outside of the cavity. The thermocouple is electrically connected to a voltmeter, a bridge or another means for measuring a voltage across the thermocouple. The measured voltage across the thermocouple is usually a DC voltage. Preferably, the thermocouple includes a first gold film adjoined to a chromium film as the sensing junction and the chromium film adjoined to a second gold film as the reference junction.

Preferably, the resistive heater and the temperature sensor are insulated from the cavity by an insulating layer. The insulating layer may be a protective layer or there may be a separate protective layer. The insulating layer or the protective layer may include SiO₂, Si₃N₄, or PTFE. The insulating layer or the protective layer may be disposed across substantially the entire first surface, and a portion of the second surface which portion is positioned opposite the well. The insulating layer or the protective layer includes a coating covering substantially all of the second surface and bottom and side surfaces of the well.

The acquisition, preparation or analysis chamber includes a cavity that may have a volume from about 0.001 μl to about 10 μl. Preferably, the cavity may have a volume from about 0.01 μl to about 1 μl, and more preferably the volume from about 0.05 μl to about 0.5 μl.

The entire process including sample acquisition, preparation or analysis may be controlled by a computer. The computer may receive signals from and provide control signals to various elements internal or external to the miniature device. These elements may include various pumps, micropumps, valves, vents, electrodes, electrical elements (including semiconducting devices) or sensors. The sensors include the above-mentioned thermocouple, or other temperature sensors, pressure sensors, volume sensors, mass sensors, chemical sensors including pH sensors, optical sensors, radioactive sensors and other sensors capable to provide signal regarding sample acquisition, preparation or analysis.

The miniature device may include at least one opening or port in communication with the sample acquisition, preparation or analysis chamber. The opening may be disposed through at least one of the first planar member or the second planar member for introducing or removing a fluid sample from the well.

The miniature device may include, in addition to at least one sample acquisition, preparation or analysis chamber, an external reaction chamber fluidly connected to, or fluidly connectable to, any internal reaction chamber (i.e., connected to or connectable to an internal sample acquisition, preparation or analysis chamber). The external reaction chamber may be a sample acquisition chamber, a preparation chamber or an analysis chamber (i.e., an analytical chamber).

The miniature device may include a micropump, a diaphragm pump, or another means for transporting a fluid sample between the internal chambers, or to and from the external chambers.

The miniature device may include one or several reservoirs. The reservoirs may include samples or one or several reaction components. Alternatively, one or several reaction components may be delivered to a reaction chamber from an external source. The one or several reaction components may include a component necessary for sample acquisition, preparation, or analysis. The reaction component may be a component necessary for a sequencing reaction, a transcription reaction, a restriction digest, a nucleic acid fragmentation reaction, or a chemical labeling reaction. The reaction component may include an effective amount of four deoxynucleoside triphosphates, a nucleic acid polymerase and amplification primer sequences.

The miniature device may include one or several mechanisms for mixing a fluid sample within or outside of the reaction chamber. The miniature device may include one or several lamb wave transducers or other transducers or wave excitation devices for mixing a fluid sample within the cavity.

According to yet another aspect, a method for processing a sample includes using any one of the above-described miniature devices.

According to yet another aspect, a method for analyzing a sample includes delivering the sample to a hybridization chamber, and providing an oligonucleotide probe array. Preferably, the method further includes one or several of the following: sample extraction, PCR amplification, nucleic acid fragmentation and labeling, extension reactions, transcription reactions, or a similar reaction. The method may further include temperature cycling of a fluid located in a reaction chamber. The method may further include degassing of a fluid located in a reaction chamber. The method may further include temperature compressing or mixing of a fluid located in a reaction chamber.

According to yet another aspect, a method of cycling a temperature of a reaction mixture for amplification of an oligonucleotide includes depositing the reaction mixture into a reaction chamber, applying electrical power to a heating element disclosed in or adjacent to the reaction chamber; cycling the application of electrical power to raise and lower the temperature of the element and thereby raising and lowering the temperature of the reaction mixture.

According to yet another aspect a monolithic integrated device includes microfluidic valves and vents, PCR amplification chambers, and capillary electrophoretic separation channels. The valves and hydrophobic vents provide controlled and sensorless sample loading into the PCR chamber. The chambers form low volume reactors that use thin film heaters. The amplified products can be labeled with an intercalating fluorescent dye and directly injected into a microfabricated capillary electrophoresis channel. Analyses with this device have produced and detected PCR products from reactions with as few as 20 starting DNA template copies/μl, (i.e., five to six copies/chamber). The extrapolated detection limit based on data using 20 cycles is two copies per chamber. The chambers make use of optimized heater placement, thermal anisotropy measurements, and optimized thermal profiles.

Preferably, miniature microfluidic devices are made using semiconductor manufacturing and other technologies. These devices include micromechanical structures such as micropumps, microvalves, microvents, microsensors and the like, incorporated into miniature chambers and flow passages.

According to yet another aspect, the miniature device is used together with a chip packaging cassette for hybridization, as described in U.S. Pat. No. 5,945,334, which is incorporated by reference.

### BRIEF DESCRIPTION OF THE DRAWINGS

FIG. 1 is a diagrammatic illustration of a processing system with several reaction chambers, reservoirs, valves, vents, pumps and sensors.

FIG. 2 illustrates a top view of a miniature integrated device that employs a centralized geometry.

FIG. 2A illustrates a side view of the device of FIG. 2, wherein the central chamber is a pumping chamber, and the device employs diaphragm valve structures for sealing individual reaction chambers.

FIG. 3 illustrates the use of pneumatic control manifolds for transporting fluid within a miniature integrated device.

FIG. 3A illustrates a manifold configuration suitable for application of negative pressure, or vacuum for moving fluids among several reaction chambers.

FIG. 4 illustrates a side sectional view of a miniaturized reactor device using a positive fluid movement scheme.

FIG. 4A illustrates a top plan view of the pneumatic portion of the reactor device of FIG. 4.

FIG. 4B illustrates a top plan view of the fluid portion of the reactor device of FIG. 4.

FIG. 5 illustrates diagrammatically a miniature integrated device having numerous reactor chambers, including degassing chamber, dosing or volumetric chamber, storage chamber, reaction chamber and other chambers.

FIG. 5A illustrates a cross-sectional view of a hybridization chamber sealed by a deformable diaphragm constructed and arranged to draw fluid into or to eject fluid from the chamber.

FIG. 5B illustrates an array of sealed pneumatic chambers located on a single device.

FIGS. 6, 6A and 6B illustrate another embodiment of the miniature device including a reaction chamber integrated into a capillary electrophoresis device. FIG. 6 illustrates a layout of a bottom substrate having microcapillary channels, reservoirs and a reaction chamber well etched into the surface, with a heater and electrical leads deposited thereon. FIG. 6A illustrates a representation of the top substrate having a thermocouple deposited thereon. FIG. 6B is a perspective view of the mating of the top and bottom substrates shown in FIGS. 6A and 6, respectively.

FIG. 7 shows a control system and power source integrating the reaction chamber of the invention.

FIG. 8 shows a mask design used to fabricate microfluidic PCR-CE chips with valves and vents.

FIGS. 9A, 9B, 9C, and 9D show the design of a vent manifold in communication with individual valves and vents for controlling fluid flow.

FIGS. 10A and 10B show a temperature profile as a function of time used for the microfluidic PCR-CE amplification and analysis

FIGS. 11A, 11B, and 11C are contour plots of the average temperature over three cycles for temperatures 95° C., 72° C., and 53° C., respectively, used in PCR amplification.

FIG. 12A presents the fluorescent results of an analysis of M13 amplicons conducted on the microfluidic PCR-CE chip.

FIG. 12B represents a positive control using the same solution amplified on a Peltier thermal cylinder as for PCR amplification measured in FIG. 12A.

FIG. 12C represents pBR322 Mspl DNA ladder for size comparison.

FIG. 13 is a plot of amplification product peak area as a function of starting template concentration.

### DESCRIPTION OF THE PREFERRED EMBODIMENTS

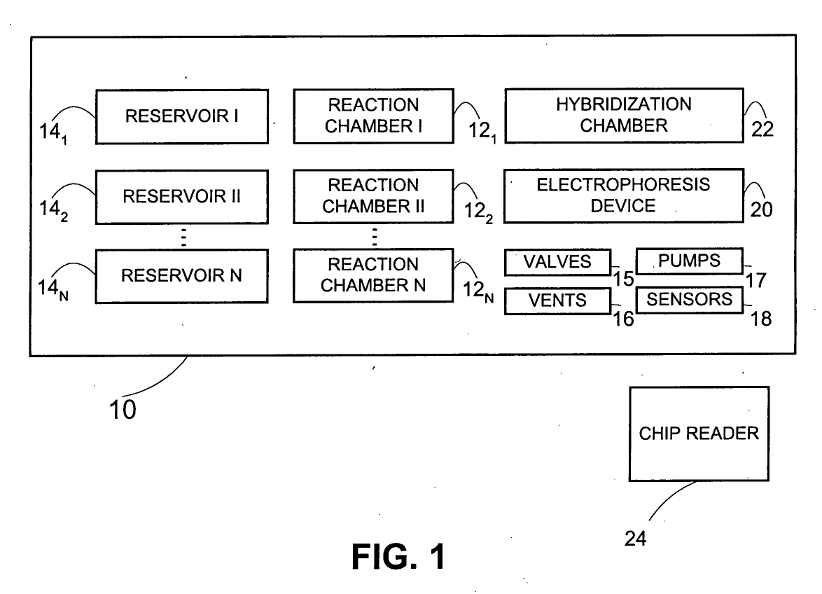

FIG. 1 illustrates diagrammatically a miniature processing device 10 including reaction chambers 12₁, 12₂, . . . , 12_{N}, reservoirs 14₁, 14₂, . . . , 14_{N}, valves 15, vents 16, pumps 17, and sensors 18. Miniature processing device 10 is constructed and arranged to perform one or several processes simultaneously or sequentially. The individual processes are used for sample preparation, processing and analysis, as described below.

Miniature processing device 10 may form an independent “lab on a chip” device or may be used together with external devices. For example, miniature processing device 10 may include an electrophoresis device 20 for analyzing the sample. Alternatively, miniature processing device 10 may include a hybridization chamber 22, which includes a probe array on a chip scanned by an external reader 24. External reader 24, for example, may be a wide field of view high speed scanning microscope described in U.S. Pat. No. 6,185,030, which is incorporated by reference. In general, miniature processing device 10 may be used with different external cartridges, readers, radiation sources and detectors, microscopes, spectrometers, and other devices.

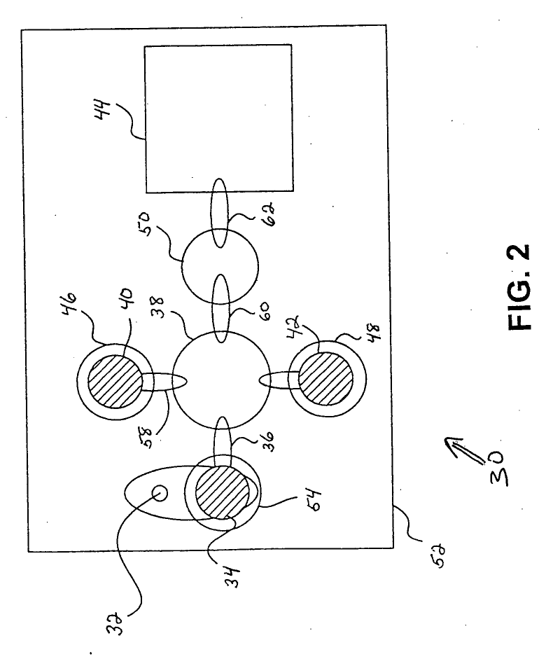

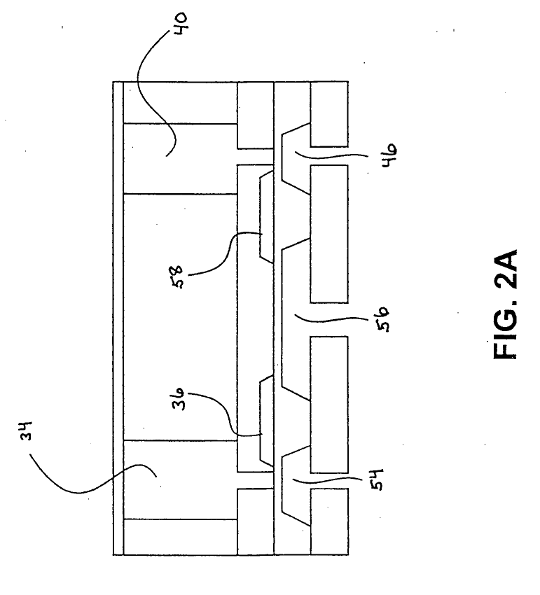

FIG. 2 illustrates a processing and analysis device 30 having several reaction chambers arranged in a centralized geometry. A central chamber 30 is constructed for gathering and distribution of a fluid sample to a number of separate collection reaction/storage/analytical chambers 34, 40, 42, 44 arranged around, and fluidly connected to central chamber 30. For example, a fluid sample is introduced into the device through sample inlet 32, which is typically fluidly connected to a sample collection chamber 34. The fluid sample is then transported to a central chamber 38 via fluid channel 36. Once within the central chamber, the sample may be transported to any one of a number of reaction/storage/analytical chambers 40, 42, 44. Each chamber 34, 40, 42 and 44, 512 and 514, includes a diaphragm 54, 46, 48 and 50, respectively, for opening and closing the fluid connection to the central chamber 30. Additional integrated reaction chambers external chambers may be added fluidly connected to the central chamber.

The central chamber may have a dual function as both a hub and a pumping chamber. In particular, this central pumping chamber can be fluidly connected to one or more additional reaction, storage or analytical chambers. This embodiment provides the advantage of a single pumping chamber to deliver a sample to numerous operations, as well as the ability to readily incorporate additional sample preparation operations within the device by opening another valve on the central pumping chamber.

In particular, central chamber 38 may incorporate a diaphragm pump as one surface of the chamber, preferably having a zero displacement when the diaphragm is not deflected. For example, the diaphragm pump will generally be fabricated from any one of a variety of flexible materials, e.g., silicon, latex, Teflon, Mylar, silicone, and the like. In particularly preferred embodiments, the diaphragm pump is silicon.

Central chamber 38 is fluidly connected to sample collection chamber 34, via fluid channel 36. Sample collection chamber is in communication with a diaphragm valve 38 for arresting fluid flow. A fluid sample is typically introduced into sample collection chamber through a sealable opening 32 in the body of the device, e.g., a valve or septum. Additionally, sample chamber 34 may incorporate a vent to allow displacement of gas or fluid during sample introduction as described in U.S. Pat. No. 6,168,948, which is incorporated by reference.

After a sample is introduced into sample collection chamber 34, it may be drawn into central pumping chamber 38 by the operation of a central diaphragm pump. Specifically, sample chamber valve 54 opens fluid channel 36 and a subsequent pulling or deflection of the central diaphragm pump creates negative pressure within pumping chamber 30, thereby drawing the sample through fluid channel 506 into central chamber 38. Subsequent closing of the sample chamber valve 54 and relaxation of the central diaphragm pump, creates a positive pressure within pumping chamber 30, which may be used to deliver the sample to additional chambers in the device.

For example, where it is desired to add specific reagents to the sample, these reagents may be stored in liquid or solid form within an adjacent storage chamber 46. Opening valve 40 opens fluid channel 58, allowing delivery of the sample into storage chamber 46 upon relaxation of the central diaphragm pump. The central pumping chamber may further be employed to mix reagents, by repeatedly pulling and pushing the sample/reagent mixture to and from the storage chamber. This has the additional advantage of eliminating the necessity of including additional mixing components within the device. Additional chamber/valve/fluid channel structures may be provided fluidly connected to pumping chamber 38 as needed to provide reagent storage chambers, additional reaction chambers or additional analytical chambers.

Referring still to FIG. 2, additional reaction/storage chamber 44 is accessible via valve 50, fluidly connected to pumping chamber 38 via fluid channel 60. Reaction chamber 44 may be used for hybridization and may be constructed for receiving an oligonucleotide probe array. Following any sample preparation operation, opening valve 50 and closure of other valves to the central pumping chamber, allows delivery of the sample through fluid channels 60 and 62 to analysis chamber 40, to the oligonucleotide array for hybridization of nucleic acids. Alternatively, analysis chamber 40 a microcapillary electrophoresis device for performing a size based analysis of the sample.

The present miniature device includes at least two miniature reaction chambers wherein the temperature of each chamber can be monitored and controlled separately. The miniaturized devices provides the benefit of low volume reactions (e.g., low sample and reagent volume requirements), high thermal transfer rates, flexibility of applications and integratability of additional functions, reproducible standardized mass production of the devices, ability to perform multiple simultaneous analyses/reactions in small spaces leading to greater automatability, and a variety of other advantages. Typically, one or several reaction chambers have a volume from about 0.001 μl to about 10 μl. Preferably, the reaction chambers have a volume from about 0.01 μl to about 1 μl, and more preferably, about 0.02 μl to about 0.5 μl.

The transportation of fluid within miniature device 30 may be carried out by a number of varied methods. For example, device 30 may use internal pump elements to transport fluid samples or reaction components between different chambers and reservoirs. Alternatively, fluid may be transported by the application of pressure differentials provided by either external or internal sources. To apply the pressure differentials, various reaction chambers of device 30 include pressure inlets for connecting reaction chambers to pressure sources (positive or negative), pressure resistances and vents.

In a first embodiment of device 30, fluid samples are moved from one reaction, storage or analytical chamber to another chamber via fluid channels by applying a positive pressure differential from an originating chamber (i.e., the chamber from which the sample is to be transported) to a receiving chamber (i.e., the chamber to which the fluid sample is to be transported). We describe initially the application of a negative pressure to the receiving chamber (but it is possible similarly to apply positive pressure, i.e., pressure to the originating chamber with only slight modifications).

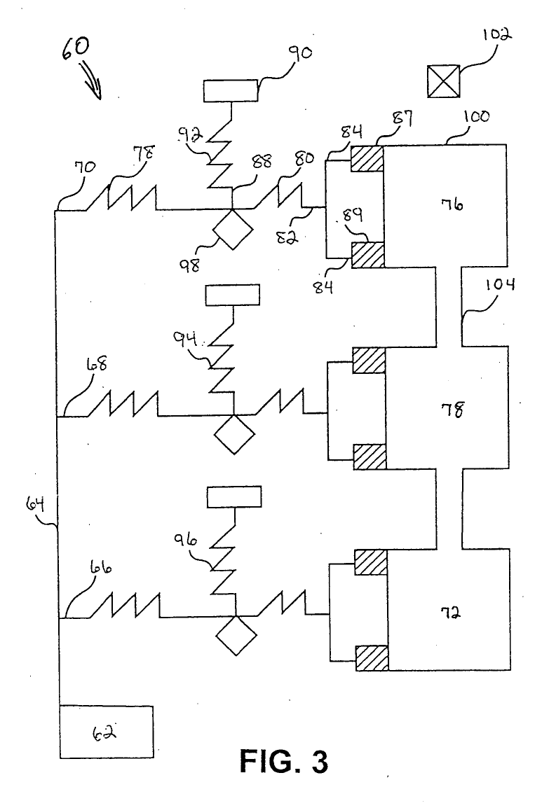

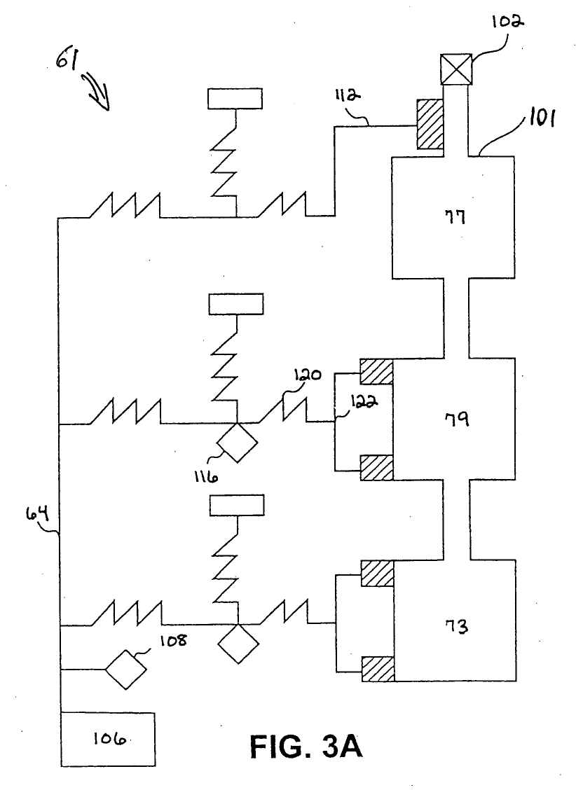

FIGS. 3 and 3A illustrate a device a pressure or vacuum manifold 60 for directing an external vacuum source to the various reaction chambers and reservoirs. Application of a pressure differential to a particular reaction chamber may generally be carried out by selectively lowering pressure in the receiving chamber. To selectively lower pressure, the reaction chamber may include an inlet with a controllable valve structure that can be selectively operated with respect to a pressure source (or a pump). Application of the pressure source to the sample chamber then forces the sample into the next reaction chamber that is at a lower pressure.

Vacuum or pressure manifold 30 produces a stepped pressure differential between each pair of connected reaction chambers. For example, assuming ambient pressure is defined as having a value of 1, a vacuum is applied to a first reaction chamber, which may be written 1−3x, where x is an incremental pressure differential. A vacuum of 1−2x is applied to a second reaction chamber, and a vacuum of 1−x is applied to a third reaction chamber in the series. Thus, the first reaction chamber is at the lowest pressure and the third is at the highest, with the second being at an intermediate level. All chambers, however, are below ambient pressure (e.g., atmospheric pressure). A sample is drawn into the first reaction chamber by the pressure differential between ambient pressure (1) and the vacuum applied to the reaction chamber (1−3x), which differential is −3x. The sample does not move to the second reaction chamber due to the pressure differential between the first and second reaction chambers (1−3x vs. 1−2x, respectively). Upon completion of the operation performed in the first reaction chamber, the vacuum is removed from the first chamber, allowing the first chamber to come to ambient pressure. At this point, the pressure differential draws the sample from the first chamber into the second chamber since there is ambient pressure in the first reaction chamber and pressure 1−2x in the second chamber. Similarly, when the operation to be performed in the second reaction chamber is completed, a vacuum source to this chamber is removed and the sample moves to the third reaction chamber.

Referring to FIG. 3, pneumatic manifold 60 for carrying out a pressure differential fluid transport includes a vacuum source 62, main vacuum channel 64, and branch channels 66, 68 and 70. Main vacuum channel 64 is connected to branch channels 66, 68 and 70, which are in turn connected to reaction chambers 72, 74 and 76, respectively, fluidly connected in series. Each branch channel includes one or more fluidic resistors 78 and 80. These fluidic resistors result in a transformation of the pressure from the pressure/vacuum source, i.e., a step down of the gas pressure or vacuum being applied across the resistance. Fluidic resistors may employ a variety of different structures. For example, a narrowing of the diameter or cross-sectional area of a channel will typically result in a fluidic resistance through the channel. Similarly, a plug within the channel which has one or more holes disposed therethrough, which effectively narrow the channel through which the pressure is applied, will result in a fluidic resistance, which resistance can be varied depending upon the number and/or size of the holes in the plug. Additionally, the plug may be fabricated from a porous material that provides a fluidic resistance through the plug, which resistance may be varied depending upon the porosity of the material and/or the number of plugs used. Variations in channel length can also be used to vary fluidic resistance.

Branch channels may be connected at a pressure nodes connected in turn to pressure inlets. Branch channel 82 is connected to reaction chamber 76 via pressure inlets 84. Pressure inlets 84 may include poorly wetting filter plugs 87 and 89, which prevent drawing of the sample into the pneumatic manifold in the case of applying vacuum. Poorly wetting filter plugs may generally be prepared from a variety of materials known in the art. Each branch channel is connected to a vent channel. For example, branch channel 70 is connected to a vent channel 88, which is opened to ambient pressure via vent 90. Vent channel 88 includes a differential fluidic resistor 92. The fluidic resistance supplied by fluidic resistor 92 is less than fluidic resistance supplied by fluidic resistor 94, which in turn is less than fluidic resistance supplied by fluidic resistor 96. As described above, this differential fluidic resistance may be accomplished by varying the diameter of the vent channel, varying the number of channels included in a single vent channel, varying channel length, or providing a plug in the vent channel having a varied number of holes disposed therethrough. Each branch channel 66, 68 or 70 connects to a sealable opening (e.g., opening 638) for introducing ambient pressure to the branch channel.

The varied fluidic resistances for each vent channel results in a varied level of vacuum being applied to each reaction chamber. For example, reaction chamber 76 may have a pressure of 1−3x, reaction chamber 78 may have a pressure of 1−2x and reaction chamber 72 may have a pressure of 1−x. The pressure of a given reaction chamber may be raised to ambient pressure. This allows the drawing of the sample into the subsequent chamber by opening the chamber to ambient pressure using the sealable opening (e.g., opening 98).

The sealable opening may include a controllable valve structure, or a rupture membrane that may be pierced at a desired time to allow the particular reaction chamber to achieve ambient pressure. Piercing of the rupture membrane may be carried out by the inclusion of solenoid operated pins incorporated within the device, or the device's base unit. In some cases, it may be desirable to prevent back flow from a previous or subsequent reaction chamber that is at a higher pressure. This may be accomplished by equipping the fluid channels between the reaction chambers with one-way check valves. Examples of one-way valve structures include ball and seat structures, flap valves, duck billed check valves, sliding valve structures, and the like.

FIG. 3A illustrates a pneumatic pressure manifold 61 for applying positive pressure to an originating chamber to push a sample into subsequent chambers. Pneumatic pressure manifold 61 includes a pressure source 106 (a pump or a pressurized vessel) which provides a positive pressure to main channel 64. Before a sample is introduced to the first reaction chamber, controllable valve 108 is opened to vent the pressure from pressure source 106. This allows the first reaction chamber 77, in the series, to remain at ambient pressure for the introduction of the sample via a sample inlet 101 having a sealable closure 102. After the sample is introduced into first reaction chamber 77, controllable valve 108 is closed, bringing system 61 up to pressure. Suitable controllable valves include any number of a variety of commercially available solenoid valves and the like. In this application, each subsequent chamber is kept at an incrementally higher pressure by the presence of the appropriate fluidic resistors and vents, as described above. A base pressure is applied at originating pressure node 112. When it is desired to deliver the sample to the second chamber 79, sealable opening 116 is opened to ambient pressure. This allows second chamber 79, to come to ambient pressure, allowing the pressure applied at the origin pressure node 112 to force the sample into the second chamber 79. Thus, illustrated as above, the first reaction chamber 77 is maintained at a pressure of 1+3x, by application of this pressure at originating pressure node 112. The second reaction chamber 79 is maintained at pressure 1+4x and the third reaction chamber 73 is maintained at a pressure of 1+5x. Opening sealable valve 116 results in a drop in the pressure of the second reaction chamber 79 to 1+2x. The pressure differential from the first to the second reaction chamber (x) pushes the sample from the first to the second reaction chamber and eventually to the third. Fluidic resistor 120 is provided between the pressure node and sealable valve 116 to prevent the escape of excess pressure when sealable valve 108 is opened. This allows system 61 to maintain a positive pressure behind the sample to push it into subsequent chambers.

A controllable pressure source may be applied to the originating reaction vessel to push a sample through the device. The pressure source is applied intermittently, as needed to move the sample from chamber to chamber. A variety of devices may be employed in applying an intermittent pressure to the originating reaction chamber (e.g., a syringe, a positive displacement pump, or the like.) Alternatively, miniature device 30 may include a thermopneumatic pump such a pump typically includes a heating element. The thermopneumatic pump includes a small scale resistive heater and a quantity of a controlled vapor pressure fluid disposed in a pressure chamber. The controlled vapor pressure fluid may include a fluorinated hydrocarbon liquid (e.g., fluorinert liquids available from 3M Corp.) having a wide range of available vapor pressures. The heater increases the controllabled temperature that in turn increases pressure in the pressure chamber. This pressure increase causes sample movement from one reaction chamber to the next. When the sample reaches the next reaction chamber, the temperature in the pressure chamber is reduced.

The above-described manifolds may include gas permeable fluid barriers, e.g., poorly wetting filter plugs or hydrophobic membranes. The gas permeable fluid barriers facilitate sensorless fluid direction and control systems for moving fluids within the device. For example, filter plugs incorporated at the end of a reaction chamber opposite a fluid inlet allow air (or other gas present in the reaction chamber) to be expelled during introduction of the fluid component into the chamber. Upon filling of the chamber, the fluid sample contacts the hydrophobic plug thus stopping net fluid flow. Fluidic resistances may also be used as gas permeable fluid barriers to accomplish this same result (e.g., using fluid passages that are sufficiently narrow as to provide an excessive fluid resistance). The resistances effectively stop or substantially retard the fluid flow while permitting air or gas flow. Expelling the fluid from the chamber then involves applying a positive pressure at the plugged vent. This permits chambers that may be filled with no valve at the inlet, i.e., to control fluid flow into the chamber. In most aspects however, a single valve will be employed at the chamber inlet in order to ensure retention of the fluid sample within the chamber, or to provide a mechanism for directing a fluid sample to one chamber of a number of chambers connected to a common channel.

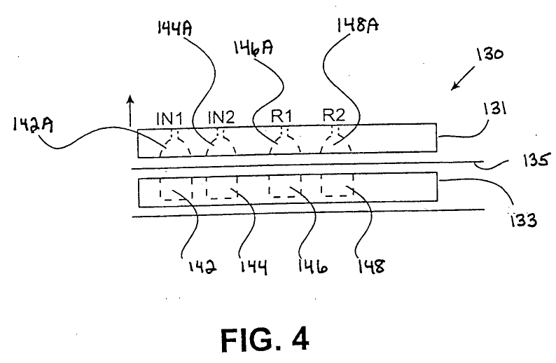

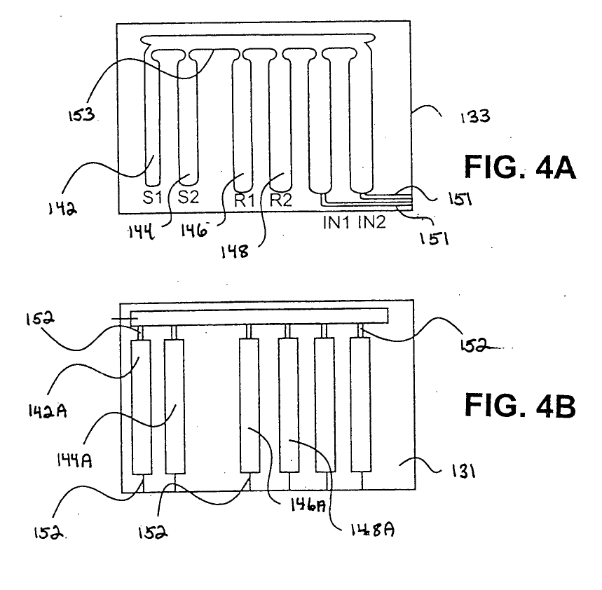

Referring to FIGS. 4, 4A, and 4B, the miniature device may include deformable reaction chambers. A deformable chamber device 130 includes a pneumatic portion 131 and a fluid portion 133 bonded together with a deformable member 135. Pneumatic portion 131 includes a plurality of reaction chambers 142, 144, 146 and 148, and fluid portion 133 includes a plurality of corresponding pneumatic chambers 142A, 144A, 146A and 148A. Chambers 142, 144, 146 and 148 include various fluid input and /or output channels 1801 (FIG. 4A) enabling fluid to enter and exit these chambers. Deformable member 1705 is preferably fabricated from polypropelene or laytex, acting as a flexible chamber wall. Pneumatic chambers 142A, 144A, 146A and 148A are positioned directly over each of reaction chambers 142, 144, 146 and 148, respectively, with deformable member 135 sealing these chambers.

As pneumatic chambers 142A, 144A, 146A and 148A are pneumatically addressed, the respective portion of deformable member 135 disposed within and thus sealing reaction chambers 142, 144, 146 and 148 moves such that the volume of these chambers can be controllably altered. Accordingly, to move fluid into a selected chamber, the pressure is decreased in its corresponding addressable port such that the deformable member moves to cause the volume of the chamber to increase. Thus, fluid can be drawn into the reaction chambers through channel 153 (FIG. 4A). Inversely, to remove fluid from a reaction chamber, the pressure is increased in its corresponding pneumatic chamber. A displacement of a portion of deformable member 135 moves to cause the volume of the chamber to decrease. Thus, fluid can be expelled from the reaction chamber through various channels 153.

In general, the above-described devices include one or several reaction chambers arranged for sequential or parallel (simultaneous) processing. Each reaction chamber may include one or several separate sensors, a heater, a thermoelectric or other cooler, and a fluid inlet that is sealed from a fluid passage by a valve. Typically, this valve can employ a variety of structures such as a flexible diaphragm structure that displaced pneumatically, magnetically or electrically. Preferably, the miniature device includes valves controlled pneumatically by applying a vacuum (or pressure) to deflect the diaphragm away from the valve seat (or push toward the valve seat), thereby creating an opening into adjoining passages (or closing a passage). Each reaction chamber may include, opposite from an inlet, an outlet vent including a porous hydrophobic membrane. The device may use a number of different commercially available hydrophobic membranes such as Versapore 200 R membranes available from Gelman Sciences. Thus fluid introduced into a reaction chamber fills the chamber until it contacts the membrane. After closing the inlet valve, the introduced fluid or several fluids are processed by mixing, heating, cooling subsequent introduction or removal of fluid to perform sample preparation, processing and analysis within the reaction chambers, as described below and in the reference publications. Each reaction chamber can be used for a separate process without being influenced by elements outside of the chamber. Furthermore, several reaction chambers can be used together to use or exchange reaction products that may then be combined or send to another reaction chamber such as a sequencing chamber or a hybridization chamber.

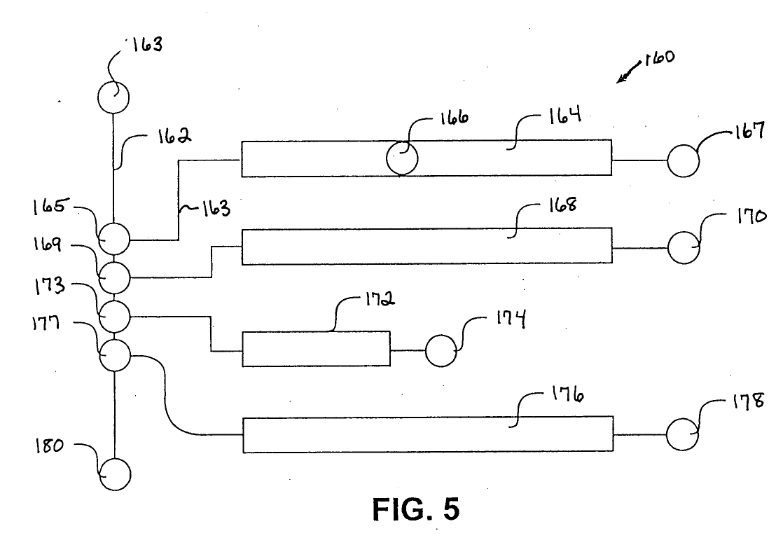

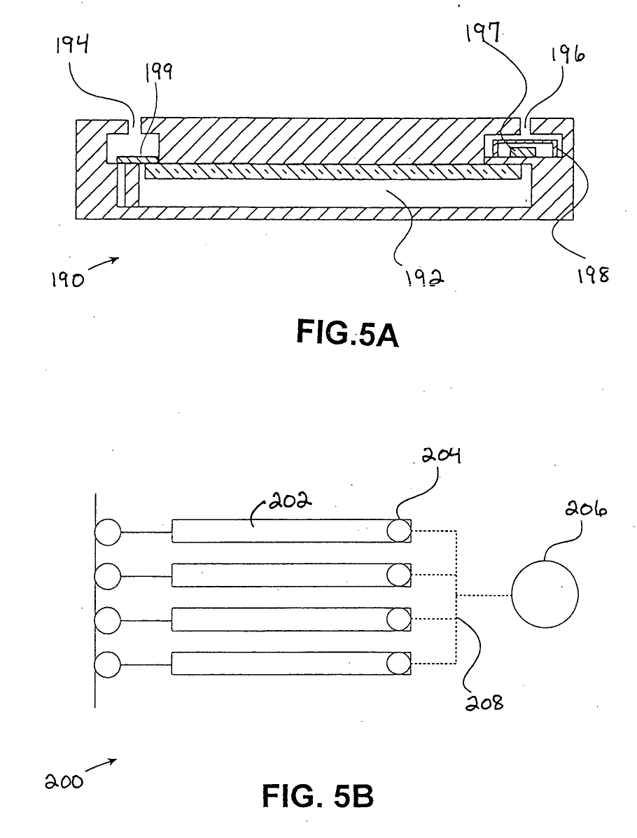

FIG. 5 illustrates diagrammatically another embodiment of the miniature device. A miniature device 160 includes a fluid flow system with a main channel 152 fluidly connected to a series of separate reaction chambers 164, 168, 172 and 174 by individual valves 165, 169, 173 and 177. Main channel 162 receives fluid via a valved or otherwise sealable liquid inlet 163 and provides fluid to reaction chamber 164 via valve 165. Main channel 162 also provides fluid to reaction chamber 168 via valve 169, to reaction chamber 172 via valve 173, and to reaction chamber 176 via valve 177. Each reaction chamber includes a vent port with a hydrophobic or poorly wetting membrane, wherein the vent port is constructed and arranged for control of fluid flow. Specifically, reaction chamber 164 includes a vent port 166, reaction chamber 168 includes a vent port 170, reaction chamber 172 includes a vent port 174, and reaction chamber 176 includes a vent port 178.

During operation, samples or other fluids may be introduced into the main channel 162 via valved or otherwise sealable fluid inlet 163 and removed via a valved or otherwise sealable fluid outlet 180. Application of a positive pressure to the fluid inlet, combined with the selective opening of one or several elastomeric valve 165, 169, 173, or 177 forces the introduced fluid into one or several chambers 164, 168, 172 or 176 and expelling of air or other gases through vent port 166, 170, 174 or 178, respectively.

For example, the individual chambers may be used for processing as follows. Referring to FIG. 5, a sample introduced into the main channel 162 is first forced into degassing chamber 164 by opening valve 165 and applying a positive pressure at inlet port 163. Until that vent is contacted with the fluid, whereupon fluid flow is stopped. The valve to the selected chamber may then be returned to the closed position to seal the fluid within the chamber. Once the fluid has filled the degassing chamber, valve 165 may then be closed. Degassing of the fluid may then be carried out by drawing a vacuum on the sample through the hydrophobic membrane disposed across the vent port 1270. Degassed sample may then be moved from the degassing chamber 164 to, e.g., reaction chamber 168, by opening valves 165 and 169, and applying a positive pressure to the degassing chamber vent port 167. The fluid is then forced from the degassing chamber 164, through main channel 162, into reaction chamber 168. When the fluid fills the reaction chamber, it will contact the hydrophobic membrane, thereby arresting fluid flow. As shown, the device includes a volumetric or measuring chamber 172 as well as a storage chamber 176, which can be used for processing. These chambers also include a similar valve and vent port arrangements for valve 173 and vent 174, and valve 177 and vent 178, respectively. The fluid may then be selectively directed to internal or external chambers as described. In short, the pressure differential needed for fluid flow may involve the application of a positive or negative pressure at a valve port or a vent port.

Furthermore, referring to FIG. 5, the above-described vents or membranes may be used for degassing or de-bubbling of fluids. For degassing purposes, for example, a chamber may include one or more vents or one wall completely or substantially bounded by a hydrophobic membrane to allow the passage of dissolved or trapped gases. Additionally, vacuum can be applied on the external surface of the membrane to draw gases from the sample fluids. Due to the small cross sectional dimensions of the reaction chambers and the fluid passages, the elimination of trapped gases takes on greater importance, as bubbles may interfere with fluid flow, or may result in production of irregular data.

According to another embodiment, one or several membranes may be used for removing bubbles purposely introduced into the device, for example, for the purpose of mixing two fluids initially desired to be separated by a bubble. For example, discrete amounts of reagents may be introduced into a single channel from several ports or reservoirs separated by a bubble. These reagents are then introduced into a reaction chamber (e.g., chamber 164), while still separated by the gas bubble that is sufficient to separate the fluids but not to inhibit fluid flow. Reaction chamber 164 includes hydrophobic membrane at vent 166. As the fluid plugs flow past the membrane, the gas will be expelled across the membrane whereupon the two fluids will mix inside chamber 164. Alternatively, a fluid channel 163 may include a vent with a hydrophobic membrane for the above-described de-bubbling and subsequent fluid mixing. Alternatively, dissolved gasses can be liberated by heating the liquid and positioning a vent along the entire length of the heating chamber.

FIGS. 5A and 5B illustrate diagrammatically another embodiment of the microfluidic device forming a hermetically sealed microfluidic system. In general, PCR reactions are extremely sensitive, but produce a high concentration of DNA product. This combination creates the danger of cross-contamination leading to erroneous results. A prior art device may, for example, contaminate an instrument through PCR-product aerosols that could find their way into subsequent tests.

The present miniaturized sample preparation device includes chambers and reservoirs for reagent storage, reactions, or hybridization. The chambers or reservoirs are separately sealable and may also be enclosed in an injection-molded package to prevent any passage of gasses or liquids between the instrument and the disposable cartridge.

FIG. 5A is an enlarged diagrammatic view of a en external reaction chamber that may be fabricated in form of a disposable cartridges 190. Disposable cartridge 190 defines a reaction chamber 192 with first and second pneumatic ports 194 and 196, respectively. Disposable cartridge 190 may include a hydrophobic vent 197, which extends between port 196 and a reaction chamber 1922. Disposable cartridge 190 may also include a deformable diaphragm seal 198, made of latex or polyimide, which covers porous hydrophobic vent 197. Fluids can be drawn into, or ejected from, the chamber by applying vacuums or pressures to the pneumatic ports 194 or 196. Diaphragm seal 198 has the desired orientation before liquid enters the reaction chamber 192 since it has only limited displacement. For example, diaphragm seal 192 is positioned in a “fully exhausted” state by pressurizing pneumatic port 196 and opening diaphragm valve 199 to eject gas into empty chamber 192. This approach can be extended to a linking or mixing chamber structures.

FIG. 5B illustrates diagrammatically a device 200 having several reaction chambers coupled to a driving chamber membrane 210. Device 200 may be a miniature device or a larger external device in form of cartridge, Device 200 includes both fluidic and pneumatic channels, vents and a pneumatic manifold. For example, a reaction chamber 202 includes a vent 204 linked to a pneumatic driving chamber 206 by an addressable pneumatic manifold 208. Pneumatic driving chamber 206 includes a driving chamber membrane appropriately positioned by exhausting gas. The driving membrane is addressed by a pneumatic port or source.

Referring to FIG. 2, hybridization of a sample to a probe array may be performed in reaction chamber 44. A nucleic acid sample, (target) can be decreased in hybridization chamber 44. Typically, aggressive mixing is necessary to achieve rapid and reproducible hybridization with sufficient signal and discrimination. One method of reducing the chamber volume is to decrease the distance between the oligonucleotide probe array and the opposite surface of the cartridge. Maintaining fluidic control while providing aggressive mixing can be challenging in this geometry because capillary forces can begin to dominate, resulting in poor convection and trapped bubbles. The present invention provides a system and method for removing bubbles and providing uniform, aggressive convection uniformly across the probe array.

Hybridization chamber 44 may include a base that defines a hybridization chamber, a pneumatic port and a fluidic port. The probe array can be mounted to the base and a thermal control block for controlling the temperature of the probe array during hybridization. A composite porous membrane can be positioned at a relatively small distance (e.g., 10 μm to 100 μm) from the probe array to create a smaller chamber therebetween. The porous membrane preferably comprises a sandwich of hydrophobic material, such as Versapore 200 from Gelman associates, and a thin membrane with neutral wetting properties, such as particle-track etched polycarbonate from Poretics.

After the target solution is introduced into the hybridization chamber, complete filling is effectively ensured by pulling a vacuum on the pneumatic port. The pneumatic port is then pressurized to inject a high density of bubbles substantially uniformly into hybridization chamber. The bubbles provide mixing by expanding, coalescing, and impacting the oligonucleotide array. Further mixing may be induced by pulling a vacuum on the pneumatic port and withdrawing the bubbles from the chamber. Alternatively, injecting and withdrawing gas from the hybridization chamber results in aggressive uniform convection to the entire oligonucleotide array surface.

Hybridization chambers with relatively small volume provide greater sensitivity and shorter assay time. The preset hybridization chambers are surface treated and may include coatings to reduce surface tension and wetting effects, thereby making the control of fluids and bubbles within the chamber possible, especially when the chamber height is small or very small, e.g. significantly below 0.5 mm.

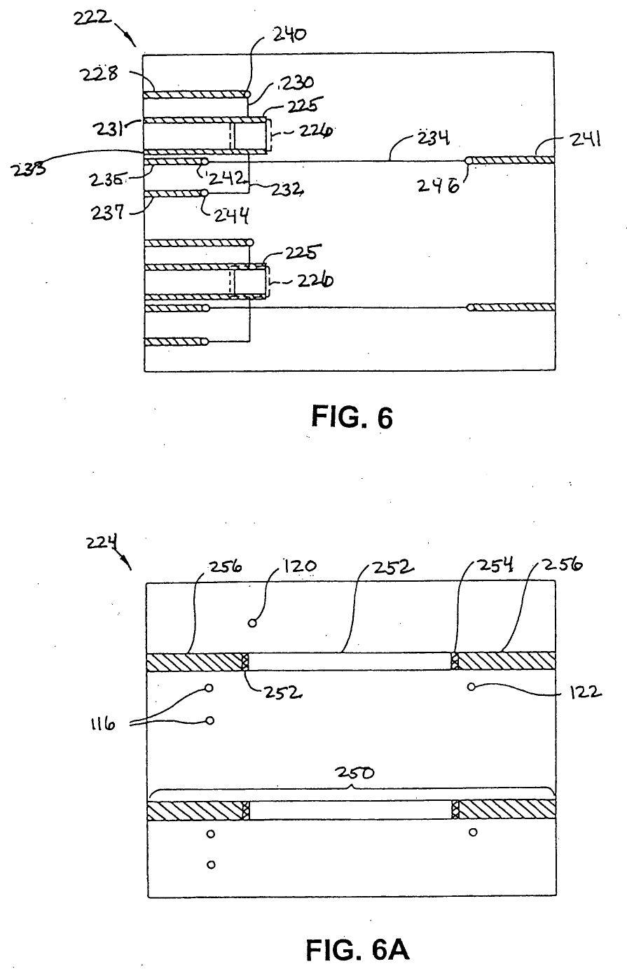

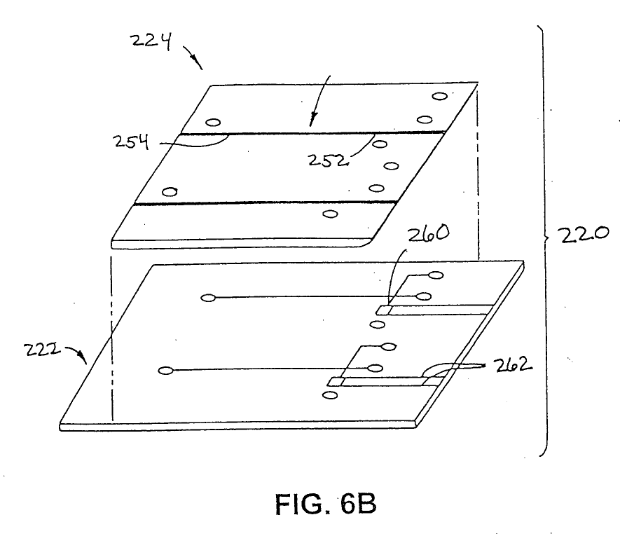

FIGS. 6, 6A and 6B illustrate another embodiment 220 of the miniature device including a reaction chamber integrated into a capillary electrophoresis device. Device 220 includes a bottom planar member 222 and a top planar member 224 (called here “substrates”, “slides” or “chips”). These planar members may be made from a variety of materials, including, e.g., plastics (press molded, injection molded, machined, etc.), glass, silicon, quartz, or other silica based materials, gallium arsenide, and the like. Preferably, at least one of the planar members is made of glass.

A reaction chamber 225 is disposed within the body of a bottom planar member 222. The cavity or well that forms the basis of the reaction chamber is generally disposed within the first planar member, and may be machined or etched into the surface. Alternatively, the cavity may be prepared in the manufacturing of the first planar member, such as where the planar member is a molded part made of plastic. The reaction chamber includes resistive heater and a thermoelectric cooler.

FIG. 6 illustrates a layout of bottom substrate 222 having microcapillary channels 230, 232 and 234, reservoirs 240, 242, and 244, and reaction chamber well 226 etched into the surface. A sample reservoir 240 receives a sample and provides it to reaction chamber 226 fluidly connected by a sample introduction channel 230. Reservoirs 242, 244 and 248 are generally filled with running buffer for the particular electrophoresis. The sample from reaction chmber 225 can be loaded in a capillary channel 232 by applying electrical current across sample reservoir 240 and buffer reservoir 244, for plug loading. The sample from reaction chamber 225 can be stack loaded by applying voltage across reservoirs 240 and 246. The application of the electrical currents across these reservoirs is done by electrical leads 228, 231, 233, 235 and 237. Following sample loading, an electrical field is applied across buffer reservoir 242 and waste reservoir 246, electrophoresing the sample through the capillary channel 234.

Device 220 includes a temperature sensor incorporated within reaction chamber 225. The temperature sensor includes a thermocouple 250 connected to and within cavity 226, and opposite a heater 260, for determination and monitoring the temperature within the reaction chamber. Thermocouple 250 includes a pair of bimetallic junctions, that is, a sensing junction 252 and a reference junction 254. Sensing junction 252 and reference junction 254 produce an electromotive force (EMF) that is proportional to the difference in the temperatures at each junction. Thermocouple 250 is connected to a device for measuring voltage across the material, e.g., a voltmeter. Thermocouple 250 is deposited on the surface of second planar member 224, and is oriented so that sensing junction 252 is electrically independent of heater 260 and its associated electrical leads 262, as illustrated in FIG. 6B.

Thermocouple 250 includes two gold/chromium junctions forming sensing and reference junctions 252 and 254, respectively, which comprises two gold strips 256 deposited on the second planar member, i.e., substrate 224. A chromium strip is then deposited to overlap the gold strips at the sensing and reference junctions (wherein the overlapping junctions are shown as double hatched regions). The gold strips of thermocouple 250 are preferably applied over a thin chromium layer, e.g., 250-350 Å thick. The gold strips themselves are preferably range in thickness of from about 2,000 Å to about 3,000 Å. The chromium element of the thermocouple is preferably from about 200 ANG. to about 4,000 Å thick.

Both thermocouple 250 and resistive heater 260 typically include an insulating layer to prevent electrical contact with the fluid sample. The insulating layer may be SiO₂ layer of from about 1,000 Å to about 4,000 Å thick.

In general the temperature sensor may also be selected from other well known miniature temperature sensing devices, such as resistance thermometers which include material having an electrical resistance proportional to the temperature of the material, thermistors, IC temperature sensors, quartz thermometers and the like. See, Horowitz and Hill, The Art of Electronics, Cambridge University Press 1994 (2nd Ed. 1994).

Resistive heater 260 (FIG. 6B) includes a thin resistive film deposited on the bottom surface of reaction well 226. Typically, the thin resistive metal film is coated with an insulating layer to prevent electrolysis at the surface of the heater, and electrophoresis of the sample components during operation. In particularly preferred embodiments, the thin metal film include a chromium film ranging in thickness from about 200 Å to about 4,000 Å, and preferably about 3,000 Å. Heater 260 is deposied by a variety of known methods, e.g., vacuum evaporation, controlled vapor deposition, sputtering, chemical decomposition methods, and the like. The protective layer over the heater includes a number of nonconductive materials, e.g., a Teflon coating, SiO₂, Si₃N₄, and the like. In particularly preferred embodiments, the heater may be coated with a SiO₂ layer. The SiO₂ layer may generally be deposited over the heater film using methods well known in the art, e.g., sputtering. Typically, this SiO₂ film will be from about 1,000 Å to about 4,000 Å thick.

Resistive heater 260 is connected to electrical leads 262, which allow the application of a voltage across the heater, and subsequent heating of the reaction chamber. A variety of conducting materials may be used as the electrical leads, however, gold leads are preferred. In particularly preferred embodiments, the electrical leads comprise a gold/chromium bilayer, having a gold layer of from about 2000 Å to about 3000 Å and a chromium layer of from about 250 Å to about 350 Å. This bilayer structure is generally incorporated to enhance the adhesion of the gold layer to the surface of the substrate. The device may use two or more heating elements or a single reaction chamber, e.g., either side of the chamber may include a heater. This design may reduce temperature gradients within the reaction chamber or across the heating element. Similarly, the heating element may be extended beyond the boundaries of the reaction chamber to accomplish the same purpose.

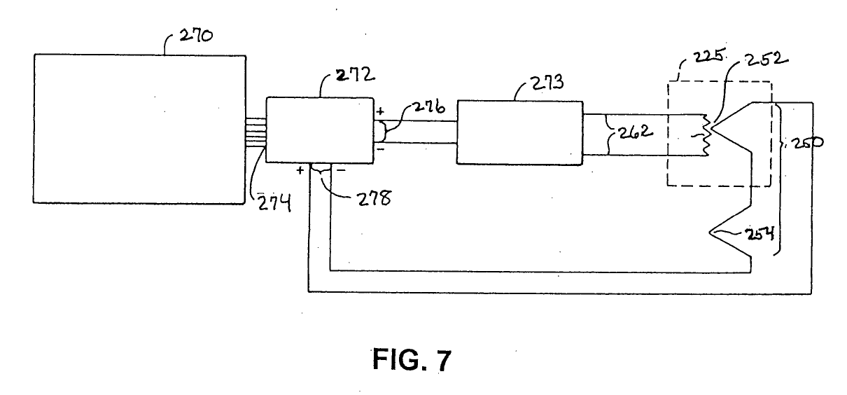

FIG. 7 illustrates computer 270 connected to an AD/DA converter 272 for monitoring thermocouple 250 and controlling heater 260. Converter 272 converts the digital signal (274) from computer 270 and provides analog output 276 to an amplifier 273. Suitable amplifiers include low power amplifiers, such as audio amplifiers, e.g., 25Vᵣₘₛ, 20 W. Amplifier is then connected via positive and negative leads 262 to the heater 260 within the reaction chamber 225. For embodiments using smaller heating elements, the voltage from the converter may be sufficient to heat the heater, thereby eliminating the need for the amplifier. Thermocouple 250 is connected to the analog input of the AD/DA converter. The EMF from the thermocouple is relayed to an analog input 278 of converter 274 and is translated to a digital signal and reported to computer 270. The computer maintains the voltage across the heater until the desired temperature is reached. When this temperature is reached within the reaction chamber, the voltage is discontinued across the heater which is then cooled by the ambient temperature surrounding the reaction chamber. When the temperature falls below the desired level, the computer again applies a voltage across the heater. The reaction chamber is generally cooled by ambient air temperature, although supplemental cooling may also be provided. Possible cooling systems include water baths, coolant systems, fans, peltier coolers, etc. Where the temperature is to be maintained at an elevated level, i.e., well above ambient temperature, the system operates as a thermostat to maintain an approximately static temperature. An AC voltage is applied across the heater, while the thermocouple provides a DC signal. This allows further differentiation between the electrical signal delivered to the heater and that received from the thermocouple by reducing the electrical “noise” measured by the thermocouple.

The miniature device includes reaction chamber 225 and additional elements for sample manipulation, transport and analysis. The reaction chambers may include openings with sealable closures that prevent leakage of the sample introduced into the chamber during operation. Sealable openings may include e.g., a silicone septum, a sealable valve, one way check valves such as flap valves or duck-billed check valves, or the like. Reaction chamber 225 may also include one or more additional elements that aid in the particular reaction or analytical operation of the reaction chamber, including, e.g., mixers, pumps, valves, vents, external irradiation sources, and the like.

Often, the convective forces resulting from the heating of a fluid sample within a reaction chamber will be sufficient to adequately mix that sample. However, in some cases it may be desirable to provide additional mixing elements. A variety of methods and devices may be employed for mixing the contents of a particular reaction chamber. For example, mixing may be carried out by applying external agitation to the reaction chamber. Typically, however, the reaction chambers of the present invention have incorporated therein, devices for mixing the contents of the reaction vessel. Examples of particularly suitable mixing methods include electro osmotic mixing, wherein the application of an electric field across the sample results in a movement of charged components within the sample and thus the mixing of the sample. Alternative suitable mixers include lamb-wave transducers, which may be incorporated into the reaction chambers, as described in PCT Publication WO 94/05414.

A number of positive displacement micropumps have been described for micron/submicron scale fluid transport including lamb-wave devices, see U.S. Pat. No. 5,006,749, electrokinetic pumps, diaphragm pumps, applied pressure differentials and the like. In particularly preferred embodiments, applied pressure differentials are used to affect fluid transport within the device, i.e., between two or more reaction chambers. In particular, the device may be provided with a pressure or vacuum manifold, as described above. The selective application of the pressure differentials can be carried out manually, i.e., by applying a vacuum or pressure to a particular reaction chamber through an opening in the chamber, or it may be carried out using a pressure manifold employing different valves according to a programmed protocol.

For a number of applications, the miniature device includes valves and vents within a given reaction chamber to accommodate reaction conditions that result in the evolution or expansion of gas or fluid within the chamber. Such vents will typically be fitted with a poorly wetting filter plug to allow for the passage of gas, while retaining liquid.

Control of reaction parameters within the reaction chamber may be carried out manually, or preferably by an appropriately programmed computer. The same computer will typically include instructions for the delivery of appropriate reagents and other fluids to the reaction chamber to follow any number of predetermined protocols, instructions for predetermined time/temperature profiles, e.g., thermal cycling for PCR, and the like.

Miniature device 220 is generally described as comprising two planar members. However, in many embodiments, each planar member may be made up of a plurality of individual elements, e.g., layers to accomplish the equivalent structure. For example, the reaction well may be formed from the mating of two substrate layers where one layer has an opening disposed therethrough. The edges of the opening will become the sides of the resulting well whereas the surface of the other substrate will become the bottom surface of the well. Furthermore, additional elements may be included within the two planar members, or may be disposed in an additional part, e.g., a third, fourth, fifth, etc. planar member. For example, flow channels may be disposed in a third planar member overlaying either the first or second member. Holes disposed through the first or second planar member can then connect these flow channels to one or more reaction chambers. This third planar member may be bonded to the reaction chamber containing body, or may be detachable, allowing rotation, or substitution with different flow channel conformations to carry out a multiplicity of varied operations. Similarly, the ability to substitute flow channel conformations can allow a single reaction chamber body to be custom fabricated to carry out any number of a variety of different applications. A third planar member may also include vacuum manifolds for operation of fluid transport systems such as pumps, valves and the like, or may include electrical circuits for operation of, or connection to the various electrical components, e.g., heaters, valves, pumps, temperature sensors, microprocessors for controlling the reaction chamber, and batteries for providing a power source for operation of these components.

In miniature device 220, reaction chamber 225 may be fluidly connected to additional reaction chambers to carry out any number of additional reactions. For example, one reaction chamber may be used to carry out a fragmentation reaction. Following this fragmentation reaction, the sample may be transported to a second reaction chamber for, e.g., PCR amplification of desired fragments, hybridization of the fragments to an array. Similarly, a first reaction chamber may be adapted for performing extension reactions, whereupon their completion, the sample may be transported to a subsequent reaction chamber for analysis, i.e., sequencing by capillary electrophoresis.

In general, the present devices are designed for the following intergated processing using miniaturized or larger size reaction chambers and channels.

1. Sample Acquisition

The sample collection portion of the device of the present invention generally provides for the identification of the sample, while preventing contamination of the sample by external elements, or contamination of the environment by the sample. Generally, this is carried out by introducing a sample for analysis, e.g., preamplified sample, tissue, blood, saliva, etc., directly into a sample collection chamber within the device. Typically, the prevention of cross-contamination of the sample may be accomplished by directly injecting the sample into the sample collection chamber through a sealable opening, e.g., an injection valve, or a septum. Generally, sealable valves are preferred to reduce any potential threat of leakage during or after sample injection. Alternatively, the device may be provided with a hypodermic needle integrated within the device and connected to the sample collection chamber, for direct acquisition of the sample into the sample chamber. This can substantially reduce the opportunity for contamination of the sample.

In addition to the foregoing, the sample collection portion of the device may also include reagents and/or treatments for neutralization of infectious agents, stabilization of the specimen or sample, pH adjustments, and the like. Stabilization and pH adjustment treatments may include, e.g., introduction of heparin to prevent clotting of blood samples, addition of buffering agents, addition of protease or nuclease inhibitors, preservatives and the like. Such reagents may generally be stored within the sample collection chamber of the device or may be stored within a separately accessible chamber, wherein the reagents may be added to or mixed with the sample upon introduction of the sample into the device. These reagents may be incorporated within the device in either liquid or lyophilized form, depending upon the nature and stability of the particular reagent used.

2. Sample Preparation

In between introducing the sample to be analyzed into the device, and analyzing that sample, e.g., on an oligonucleotide array, it will often be desirable to perform one or more sample preparation operations upon the sample. Typically, these sample preparation operations will include such manipulations as extraction of intracellular material, e.g., nucleic acids from whole cell samples, viruses and the like, amplification of nucleic acids, fragmentation, transcription, labeling and/or extension reactions. One or more of these various operations may be readily incorporated into the device of the present invention.

3. Nucleic Acid Extraction

For those embodiments where whole cells, viruses or other tissue samples are being analyzed, it will typically be necessary to extract the nucleic acids from the cells or viruses, prior to continuing with the various sample preparation operations. Accordingly, following sample collection, nucleic acids may be liberated from the collected cells, viral coat, etc., into a crude extract, followed by additional treatments to prepare the sample for subsequent operations, e.g., denaturation of contaminating (DNA binding) proteins, purification, filtration, desalting, and the like.

Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within the extraction chamber, a separate accessible chamber or externally introduced.

Alternatively, physical methods may be used to extract the nucleic acids and denature DNA binding proteins. U.S. Pat. No. 5,304,487, incorporated herein by reference in its entirety for all purposes, discusses the use of physical protrusions within microchannels or sharp edged particles within a chamber or channel to pierce cell membranes and extract their contents. Combinations of such structures with piezoelectric elements for agitation can provide suitable shear forces for lysis. Such elements are described in greater detail with respect to nucleic acid fragmentation, below. More traditional methods of cell extraction may also be used, e.g., employing a channel with restricted cross-sectional dimension which causes cell lysis when the sample is passed through the channel with sufficient flow pressure.

Alternatively, cell extraction and denaturing of contaminating proteins may be carried out by applying an alternating electrical current to the sample. More specifically, the sample of cells is flowed through a microtubular array while an alternating electric current is applied across the fluid flow. A variety of other methods may be utilized within the device of the present invention to effect cell lysis/extraction, including, e.g., subjecting cells to ultrasonic agitation, or forcing cells through microgeometry apertures, thereby subjecting the cells to high shear stress resulting in rupture.

Following extraction, it will often be desirable to separate the nucleic acids from other elements of the crude extract, e.g., denatured proteins, cell membrane particles, salts, and the like. Removal of particulate matter is generally accomplished by filtration, flocculation or the like. A variety of filter types may be readily incorporated into the device. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample, passing salts through dialysis membranes, and the like. Suitable solid supports for nucleic acid binding include, e.g., diatomaceous earth, silica (i.e., glass wool), or the like. Suitable gel exclusion media, also well known in the art, may also be readily incorporated into the devices of the present invention, and is commercially available from, e.g., Pharmacia and Sigma Chemical.

The isolation and/or gel filtration/desalting may be carried out in an additional chamber, or alternatively, the particular chromatographic media may be incorporated in a channel or fluid passage leading to a subsequent reaction chamber. Alternatively, the interior surfaces of one or more fluid passages or chambers may themselves be derivatized to provide functional groups appropriate for the desired purification, e.g., charged groups, affinity binding groups and the like, i.e., poly-T oligonucleotides for mRNA purification.

Alternatively, desalting methods may generally take advantage of the high electrophoretic mobility and negative charge of DNA compared to other elements. Electrophoretic methods may also be utilized in the purification of nucleic acids from other cell contaminants and debris. In one example, a separation channel or chamber of the device is fluidly connected to two separate “field” channels or chambers having electrodes, e.g., platinum electrodes, disposed therein. The two field channels are separated from the separation channel using an appropriate barrier or “capture membrane” which allows for passage of current without allowing passage of nucleic acids or other large molecules. The barrier generally serves two basic functions: first, the barrier acts to retain the nucleic acids which migrate toward the positive electrode within the separation chamber; and second, the barriers prevent the adverse effects associated with electrolysis at the electrode from entering into the reaction chamber (e.g., acting as a salt junction). Such barriers may include, e.g., dialysis membranes, dense gels, PEI filters, or other suitable materials. Upon application of an appropriate electric field, the nucleic acids present in the sample will migrate toward the positive electrode and become trapped on the capture membrane. Sample impurities remaining free of the membrane are then washed from the chamber by applying an appropriate fluid flow. Upon reversal of the voltage, the nucleic acids are released from the membrane in a substantially purer form. The field channels may be disposed on the same or opposite sides or ends of a separation chamber or channel, and may be used in conjunction with mixing elements described herein, to ensure maximal efficiency of operation. Further, coarse filters may also be overlaid on the barriers to avoid any fouling of the barriers by particulate matter, proteins or nucleic acids, thereby permitting repeated use.

In a similar aspect, the high electrophoretic mobility of nucleic acids with their negative charges, may be utilized to separate nucleic acids from contaminants by utilizing a short column of a gel or other appropriate matrix or gel which will slow or retard the flow of other contaminants while allowing the faster nucleic acids to pass.

For a number of applications, it may be desirable to extract and separate messenger RNA from cells, cellular debris, and other contaminants. As such, the device of the present invention may, in some cases, include an mRNA purification chamber or channel. In general, such purification takes advantage of the poly-A tails on mRNA. In particular and as noted above, poly-T oligonucleotides may be immobilized within a chamber or channel of the device to serve as affinity ligands for mRNA. Poly-T oligonucleotides may be immobilized upon a solid support incorporated within the chamber or channel, or alternatively, may be immobilized upon the surface(s) of the chamber or channel itself. Immobilization of oligonucleotides on the surface of the chambers or channels may be carried out by methods described herein including, e.g., oxidation and silanation of the surface followed by standard DMT synthesis of the oligonucleotides.

In operation, the lysed sample is introduced into this chamber or channel in an appropriate salt solution for hybridization, whereupon the mRNA will hybridize to the immobilized poly-T. Hybridization may also be enhanced through incorporation of mixing elements, also as described herein. After enough time has elapsed for hybridization, the chamber or channel is washed with clean salt solution. The mRNA bound to the immobilized poly-T oligonucleotides is then washed free in a low ionic strength buffer. The surface area upon which the poly-T oligonucleotides are immobilized may be increased through the use of etched structures within the chamber or channel, e.g., ridges, grooves or the like. Such structures also aid in the agitation of the contents of the chamber or channel, as described herein. Alternatively, the poly-T oligonucleotides may be immobilized upon porous surfaces, e.g., porous silicon, zeolites, silica xerogels, cellulose, sintered particles, or other solid supports.

4. PCR Amplification and In Vitro Transcription

Following sample collection and nucleic acid extraction, the nucleic acid portion of the sample is typically subjected to one or more preparative reactions. These preparative reactions include in vitro transcription, labeling, fragmentation, amplification and other reactions. Nucleic acid amplification increases the number of copies of the target nucleic acid sequence of interest. A variety of amplification methods are suitable for use in the methods and device of the present invention, including for example, the polymerase chain reaction method or (PCR), the ligase chain reaction (LCR), self sustained sequence replication (3SR), and nucleic acid based sequence amplification (NASBA).

The latter two amplification methods involve isothermal reactions based on isothermal transcription, which produce both single stranded RNA (ssRNA) and double stranded DNA (dsDNA) as the amplification products in a ratio of approximately 30 or 100 to 1, respectively. As a result, where these latter methods are employed, sequence analysis may be carried out using either type of substrate, i.e., complementary to either DNA or RNA.

In particularly preferred aspects, the amplification step is carried out using PCR techniques that are well known in the art. See PCR Protocols: A Guide to Methods and Applications (Innis, M., Gelfand, D., Sninsky, J. and White, T., eds.) Academic Press (1990), incorporated herein by reference in its entirety for all purposes. PCR amplification generally involves the use of one strand of the target nucleic acid sequence as a template for producing a large number of complements to that sequence. Generally, two primer sequences complementary to different ends of a segment of the complementary strands of the target sequence hybridize with their respective strands of the target sequence, and in the presence of polymerase enzymes and deoxy-nucleoside triphosphates, the primers are extended along the target sequence. The extensions are melted from the target sequence and the process is repeated, this time with the additional copies of the target sequence synthesized in the preceding steps. PCR amplification typically involves repeated cycles of denaturation, hybridization and extension reactions to produce sufficient amounts of the target nucleic acid. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In PCR methods, strand separation is normally achieved by heating the reaction to a sufficiently high temperature for a sufficient time to cause the denaturation of the duplex but not to cause an irreversible denaturation of the polymerase enzyme (see U.S. Pat. No. 4,965,188, incorporated herein by reference). Typical heat denaturation involves temperatures ranging from about 80 degree Celsius to 105 degree Celsius for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference). Other embodiments may achieve strand separation by application of electric fields across the sample. For example, Published PCT Application Nos. WO 92/04470 and WO 95/25177, incorporated herein by reference, describe electrochemical methods of denaturing double stranded DNA by application of an electric field to a sample containing the DNA. Structures for carrying out this electrochemical denaturation include a working electrode, counter electrode and reference electrode arranged in a potentiostat arrangement across a reaction chamber (See, Published PCT Application Nos. WO 92/04470 and WO 95/25177, each of which is incorporated herein by reference for all purposes). Such devices may be readily miniaturized for incorporation into the devices of the present invention utilizing the microfabrication techniques described herein.

Template-dependent extension of primers in PCR is catalyzed by a polymerizing agent in the presence of adequate amounts of at least 4 deoxyribonucleotide triphosphates (typically selected from DATP, dGTP, dCTP, dUTP and dTTP) in a reaction medium which comprises the appropriate salts, metal cations, and pH buffering system. Reaction components and conditions are well known in the art (See PCR Protocols: A Guide to Methods and Applications (Innis, M., Gelfand, D., Sninsky, J. and White, T., eds.) Academic Press (1990), previously incorporated by reference). Suitable polymerizing agents are enzymes known to catalyze template-dependent DNA synthesis.

Published PCT Application No. WO 94/05414, to Northrup and White, discusses the use of a microPCR chamber which incorporates microheaters and micropumps in the thermal cycling and mixing during the PCR reactions.

The amplification reaction chamber of the device may comprise a sealable opening for the addition of the various amplification reagents. However, in preferred aspects, the amplification chamber will have an effective amount of the various amplification reagents described above, predisposed within the amplification chamber, or within an associated reagent chamber whereby the reagents can be readily transported to the amplification chamber upon initiation of the amplification operation. By “effective amount” is meant a quantity and/or concentration of reagents required to carry out amplification of a targeted nucleic acid sequence. These amounts are readily determined from known PCR protocols. See, e.g., Sambrook, et al. Molecular Cloning: A Laboratory Manual, (2nd ed.) Vols. 1-3, Cold Spring Harbor Laboratory, (1989) and PCR Protocols: A Guide to Methods and Applications (Innis, M., Gelfand, D., Sninsky, J. and White, T., eds.) Academic Press (1990), both of which are incorporated herein by reference for all purposes in their entirety. For those embodiments where the various reagents are predisposed within the amplification or adjacent chamber, it will often be desirable for these reagents to be in lyophilized forms, to provide maximum shelf life of the overall device. Introduction of the liquid sample to the chamber then reconstitutes the reagents in active form, and the particular reactions may be carried out.

In some aspects, the polymerase enzyme may be present within the amplification chamber, coupled to a suitable solid support, or to the walls and surfaces of the amplification chamber. Suitable solid supports include those that are well known in the art, e.g., agarose, cellulose, silica, divinylbenzene, polystyrene, etc. Coupling of enzymes to solid supports has been reported to impart stability to the enzyme in question, which allows for storage of days, weeks or even months without a substantial loss in enzyme activity, and without the necessity of lyophilizing the enzyme. The 94 kd, single subunit DNA polymerase from Thermus aquaticus (or taq polymerase) is particularly suited for the PCR based amplification methods used in the present invention, and is generally commercially available from, e.g., Promega, Inc., Madison, Wis. In particular, monoclonal antibodies are available which bind the enzyme without affecting its polymerase activity. Consequently, covalent attachment of the active polymerase enzyme to a solid support, or the walls of the amplification chamber can be carried out by using the antibody as a linker between the enzyme and the support.

In addition to PCR and IVT reactions, the methods and devices of the present invention are also applicable to a number of other reaction types, e.g., reverse transcription, nick translation, cDNAse generation, and the like.

In one embodiment, acoustic microstructures may be used for hybridization mixing. A description of an acoustic mixer may be found in X. Zhu and E. S. Kim “Microfluidic Motion Generation With Loosely-Focused Acoustic Waves”, 1997 Int'l. Conference on Solid-State Sensors and Actuators, Jun. 16-19, 1997, Chicago, Ill.

5. Labeling and Fragmentation

The nucleic acids in a sample will generally be labeled to facilitate detection in subsequent steps. Labeling may be carried out during the amplification, in vitro transcription or nick translation processes. In particular, amplification, in vitro transcription or nick translation may incorporate a label into the amplified or transcribed sequence, either through the use of labeled primers or the incorporation of labeled dNTPs or NTPs into the amplified sequence. Labeling may also be carried out by attaching an appropriately labeled (e.g. FICT, or biotin), dNTP to the 3′-end of DNAase fragmented PCR product using terminal deoxy-transferase (TdT).

In an alternative embodiment, Poly(A) polymerase will “tail” any RNA molecule with polyA and therefore be used for radiolabeling RNA. Used in conjunction with a biotin-, fluorophore-, gold particle—(or other detectable moiety)—ATP conjugate, poly (A) polymerase can be used for direct 3′-end labelling of RNA targets for detecting hybridization to DNA probe arrays. The nucleotide conjugate may carry the detectable moiety attached, through a linker (or not) to positions on either the nucleotide base or sugar. With regard to relative incorporation efficiency, the enzyme may exhibit a preference for one or more of these positions. The nucleotide may be a 2′, 3′-dideoxynucleotide, in which case only a single label will be added to the 3′-end of the RNA. A preferred format is to tail the RNA with 5-Bromo-UTP, and then detect hybridization indirectly using a labeled anti-bromouridine. This would closely parallel a currently favored assay format used for expression monitoring applications using biotinylated RNA and phycoerythrin-streptavidin “staining”.

Alternatively, the nucleic acids in the sample may be labeled following amplification. Post amplification labeling typically involves the covalent attachment of a particular detectable group upon the amplified sequences. Suitable labels or detectable groups include a variety of fluorescent or radioactive labeling groups well known in the art. These labels may also be coupled to the sequences using methods that are well known in the art. See, e.g., Sambrook, et al.

In addition, amplified sequences may be subjected to other post amplification treatments. For example, in some cases, it may be desirable to fragment the sequence prior to hybridization with an oligonucleotide array, in order to provide segments which are more readily accessible to the probes, which avoid looping and/or hybridization to multiple probes. Fragmentation of the nucleic acids may generally be carried out by physical, chemical or enzymatic methods that are known in the art. These additional treatments may be performed within the amplification chamber, or alternatively, may be carried out in a separate chamber. For example, physical fragmentation methods may involve moving the sample containing the nucleic acid over pits or spikes in the surface of a reaction chamber or fluid channel. The motion of the fluid sample, in combination with the surface irregularities produces a high shear rate, resulting in fragmentation of the nucleic acids. In one aspect, this may be accomplished in a miniature device by placing a piezoelectric element, e.g., a PZT ceramic element adjacent to a substrate layer that covers a reaction chamber or flow channel, either directly, or through a liquid layer, as described herein. The substrate layer has pits, spikes or apertures manufactured in the surface which are within the chamber or flow channel. By driving the PZT element in the thickness mode, a standing wave is set up within the chamber. Cavitation and/or streaming within the chamber results in substantial shear. Similar shear rates may be achieved by forcing the nucleic acid containing fluid sample through restricted size flow passages, e.g., apertures having a cross-sectional dimension in the micron or submicron scale, thereby producing a high shear rate and fragmenting the nucleic acid.

A number of sample preparation operations may be carried out by adjusting the pHof the sample, such ascelllysis, nucleic acid fragmentation, enzyme denaturation and the like. Similarly, pH control may also play a role in a wide variety of other reactions to be carried out in the device, i.e., for optimizing reaction conditions, neutralizing acid or base additions, denaturing exogenously introduced enzymes, quenching reactions, and the like. Such pH monitoring and control may be readily accomplished using well known methods. For example, pH may be monitored by incorporation of a pH sensor or indicator within a particular chamber. Control may then be carried out by titration of the chamber contents with an appropriate acid or base.

Fragmentation may also be carried out enzymatically using, for example, DNAase or RNAase or restriction enzymes.

6. Sample Analysis

Following the various sample preparation operations, the sample will generally be subjected to one or more analysis operations. Particularly preferred analysis operations include, e.g., sequence based analyses using an oligonucleotide array and/or size based analyses using, e.g., microcapillary array electrophoresis.

A. Capillary Electrophoresis

In some embodiments, it may be desirable to provide an additional, or alternative means for analyzing the nucleic acids from the sample. In one embodiment, the device of the invention will optionally or additionally comprise a micro capillary array for analysis of the nucleic acids obtained from the sample.

Microcapillary array electrophoresis generally involves the use of a thin capillary or channel which may or may not be filled with a particular separation medium. Electrophoresis of a sample through the capillary provides a size based separation profile for the sample. The use of microcapillary electrophoresis in size separation of nucleic acids has been reported in, e.g., Woolley and Mathies, Proc. Nat'l Acad. Sci. USA (1994) 91:11348-11352. Microcapillary array electrophoresis generally provides a rapid method for size based sequencing, PCR product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods.

Microfabrication of microfluidic devices including microcapillary electrophoretic devices has been discussed in detail in, e.g., Jacobsen, et al., Anal. Chem. (1994) 66:1114-1118, Effenhauser, et al., Anal. Chem. (1994) 66:2949-2953, Harrison, et al., Science (1993) 261:895-897, Effenhauser, et al. Anal. Chem. (1993) 65:2637-2642, and Manz, et al., J. Chromatog. (1992) 593:253-258. Typically, these methods comprise photolithographic etching of micron scale channels on a silica, silicon or other rigid substrate or chip, and can be readily adapted for use in the miniaturized devices of the present invention. In some embodiments, the capillary arrays may be fabricated from the same polymeric materials described for the fabrication of the body of the device, using the injection molding techniques described herein. In such cases, the capillary and other fluid channels may be molded into a first planar element. A second thin polymeric member having ports corresponding to the termini of the capillary channels disposed therethrough, is laminated or sonically welded onto the first to provide the top surface of these channels. Electrodes for electrophoretic control are disposed within these ports/wells for application of the electrical current to the capillary channels. Through use of a relatively this sheet as the covering member of the capillary channels, heat generated during electrophoresis can be rapidly dissipated. Additionally, the capillary channels may be coated with more thermally conductive material, e.g., glass or ceramic, to enhance heat dissipation.

In many capillary electrophoresis methods, the capillaries, e.g., fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g., hydroxyethyl cellulose, polyacrylamide, agarose and the like. Gel matrices may be introduced and polymerized within the capillary channel. However, in some cases, this may result in entrapment of bubbles within the channels which can interfere with sample separations. Accordingly, it is often desirable to place a preformed separation matrix within the capillary channel(s), prior to mating the planar elements of the capillary portion. Fixing the two parts, e.g., through sonic welding, permanently fixes the matrix within the channel. Polymerization outside of the channels helps to ensure that no bubbles are formed. Further, the pressure of the welding process helps to ensure a void-free system. Generally, the specific gel matrix, running buffers and running conditions are selected to maximize the separation characteristics of the particular application, e.g., the size of the nucleic acid fragments, the required resolution, and the presence of native or undenatured nucleic acid molecules. For example, running buffers may include denaturants, chaotropic agents such as urea or the like, to denature nucleic acids in the sample.

In addition to its use in nucleic acid “fingerprinting” and other sized based analyses, the capillary arrays may also be used in sequencing applications. In particular, gel based sequencing techniques may be readily adapted for capillary array electrophoresis. For example, capillary electrophoresis may be combined with the Sanger dideoxy chain termination sequencing methods as discussed in Sambrook, et al. (See also Brenner, et al., Proc. Nat'l Acad. Sci. (1989) 86:8902-8906). In these methods, the sample nucleic acid is amplified in the presence of fluorescent dideoxynucleoside triphosphates in an extension reaction. The random incorporation of the dideoxynucleotides terminates transcription of the nucleic acid. This results in a range of transcription products differing from another member by a single base. Comparative size based separation then allows the sequence of the nucleic acid to be determined based upon the last dideoxy nucleotide to be incorporated.

B. Oligonucleotide Probe Array

In one aspect, following sample preparation, the nucleic acid sample is probed using an array of oligonucleotide probes. Oligonucleotide arrays generally include a substrate having a large number of positionally distinct oligonucleotide probes attached to the substrate. These oligonucleotide arrays, also described as “Genechip.TM. arrays,” have been generally described in the art, for example, U.S. Pat. No. 5,143,854 and PCT patent publication Nos. WO 90/15070 and 92/10092. These pioneering arrays may be produced using mechanical or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092, all incorporated herein by reference. These references disclose methods of forming vast arrays of peptides, oligonucleotides and other polymer sequences using, for example, light-directed synthesis techniques. Techniques for the synthesis of these arrays using mechanical synthesis strategies are described in, e.g., PCT Publication No. 93/09668 and U.S. Pat. No. 5,384,261, each of which is incorporated herein by reference in its entirety for all purposes. Incorporation of these arrays in injection molded polymeric casings has been described in Published PCT Application No. 95/33846.

The basic strategy for light directed synthesis of oligonucleotide arrays is as follows. The surface of a solid support, modified with photosensitive protecting groups is illuminated through a photolithographic mask, yielding reactive hydroxyl groups in the illuminated regions. A selected nucleotide, typically in the form of a 3′-O-phosphoramidite-activated deoxynucleoside (protected at the 5′ hydroxyl with a photosensitive protecting group), is then presented to the surface and coupling occurs at the sites that were exposed to light. Following capping and oxidation, the substrate is rinsed and the surface is illuminated through a second mask, to expose additional hydroxyl groups for coupling. A second selected nucleotide (e.g., 5′-protected, 3′-O-phosphoramidite-activated deoxynucleoside) is presented to the surface. The selective deprotection and coupling cycles are repeated until the desired set of products is obtained. Since photolithography is used, the process can be readily miniaturized to generate high density arrays of oligonucleotide probes. Furthermore, the sequence of the oligonucleotides at each site is known, see, Pease, et al. Mechanical synthesis methods are similar to the light directed methods except involving mechanical direction of fluids for deprotection and addition in the synthesis steps.

Typically, the arrays used in the present invention will have a site density of greater than 100 different probes per cm.sup.2. Preferably, the arrays will have a site density of greater than 500/cm.sup.2, more preferably greater than about 1000/cm.sup.2, and most preferably, greater than about 10,000/cm.sup.2. Preferably, the arrays will have more than 100 different probes on a single substrate, more preferably greater than about 1000 different probes still more preferably, greater than about 10,000 different probes and most preferably, greater than 100,000 different probes on a single substrate.

For some embodiments, oligonucleotide arrays may be prepared having all possible probes of a given length. Such arrays may be used in such areas as sequencing or sequence checking applications, which offer substantial benefits over traditional methods. The use of oligonucleotide arrays in such applications is described in, e.g., U.S. patent application Ser. No. 08/505,919, filed Jul. 24, 1995, now abandoned, and U.S. patent application Ser. No. 08/284,064, filed Aug. 2, 1994, now abandoned, each of which is incorporated herein by reference in its entirety for all purposes. These methods typically use a set of short oligonucleotide probes of defined sequence to search for complementary sequences on a longer target strand of DNA. The hybridization pattern of the target sequence on the array is used to reconstruct the target DNA sequence. Hybridization analysis of large numbers of probes can be used to sequence long stretches of DNA.

One strategy of de novo sequencing can be illustrated by the following example. A 12-mer target DNA sequence is probed on an array having a complete set of octanucleotide probes. Five of the 65,536 octamer probes will perfectly hybridize to the target sequence. The identity of the probes at each site is known. Thus, by determining the locations at which the target hybridizes on the array, or the hybridization pattern, one can determine the sequence of the target sequence. While these strategies have been proposed and utilized in some applications, there has been difficulty in demonstrating sequencing of larger nucleic acids using these same strategies. Accordingly, in preferred aspects, SBH methods utilizing the devices described herein use data from mismatched probes, as well as perfectly matching probes, to supply useful sequence data, as described in U.S. patent application Ser. No. 08/505,919, now abandoned, incorporated herein by reference.

While oligonucleotide probes may be prepared having every possible sequence of length n, it will often be desirable in practicing the present invention to provide an oligonucleotide array which is specific and complementary to a particular nucleic acid sequence. For example, in particularly preferred aspects, the oligonucleotide array will contain oligonucleotide probes which are complementary to specific target sequences, and individual or multiple mutations of these. Such arrays are particularly useful in the diagnosis of specific disorders which are characterized by the presence of a particular nucleic acid sequence. For example, the target sequence may be that of a particular exogenous disease causing agent, e.g., human immunodeficiency virus (see, U.S. application Ser. No. 08/284,064, now abandoned, previously incorporated herein by reference), or alternatively, the target sequence may be that portion of the human genome which is known to be mutated in instances of a particular disorder, i.e., sickle cell anemia (see, e.g., U.S. application Ser. No. 08/082,937, now abandoned, previously incorporated herein by reference) or cystic fibrosis.

In such an application, the array generally comprises at least four sets of oligonucleotide probes, usually from about 9 to about 21 nucleotides in length. A first probe set has a probe corresponding to each nucleotide in the target sequence. A probe is related to its corresponding nucleotide by being exactly complementary to a subsequence of the target sequence that includes the corresponding nucleotide. Thus, each probe has a position, designated an interrogation position, that is occupied by a complementary nucleotide to the corresponding nucleotide in the target sequence. The three additional probe sets each have a corresponding probe for each probe in the first probe set, but substituting the interrogation position with the three other nucleotides. Thus, for each nucleotide in the target sequence, there are four corresponding probes, one from each of the probe sets. The three corresponding probes in the three additional probe sets are identical to the corresponding probe from the first probe or a subsequence thereof that includes the interrogation position, except that the interrogation position is occupied by a different nucleotide in each of the four corresponding probes.

Some arrays have fifth, sixth, seventh and eighth probe sets. The probes in each set are selected by analogous principles to those for the probes in the first four probe sets, except that the probes in the fifth, sixth, seventh and eighth sets exhibit complementarity to a second reference sequence. In some arrays, the first set of probes is complementary to the coding strand of the target sequence while the second set is complementary to the noncoding strand. Alternatively, the second reference sequence can be a subsequence of the first reference sequence having a substitution of at least one nucleotide.

In some applications, the target sequence has a substituted nucleotide relative to the probe sequence in at least one undetermined position, and the relative specific binding of the probes indicates the location of the position and the nucleotide occupying the position in the target sequence.

Following amplification and/or labeling, the nucleic acid sample is incubated with the oligonucleotide array in the hybridization chamber. Hybridization between the sample nucleic acid and the oligonucleotide probes upon the array is then detected, using, e.g., epifluorescence confocal microscopy. Typically, sample is mixed during hybridization to enhance hybridization of nucleic acids in the sample to nucleic acid probes on the array. Again, mixing may be carried out by the methods described herein, e.g., through the use of piezoelectric elements, electrophoretic methods, or physical mixing by pumping fluids into and out of the hybridization chamber, i.e., into an adjoining chamber. Generally, the detection operation will be performed using a reader device external to the diagnostic device. However, it may be desirable in some cases, to incorporate the data gathering operation into the diagnostic device itself. Novel systems for direct electronic detection of hybridization locations on the array will be set forth herein.

The hybridization data is next analyzed to determine the presence or absence of a particular sequence within the sample, or by analyzing multiple hybridizations to determine the sequence of the target nucleic acid using the SBH techniques already described.

In some cases, hybridized oligonucleotides may be labeled following hybridization. For example, where biotin labeled dNTPs are used in, e.g., amplification or transcription, streptavidin linked reporter groups may be used to label hybridized complexes. Such operations are readily integratable into the systems of the present invention, requiring the use of various mixing methods as is necessary.

Gathering data from the various analysis operations, e.g., oligonucleotide and/or microcapillary arrays, will typically be carried out using methods known in the art. For example, the arrays may be scanned using lasers to excite fluorescently labeled targets that have hybridized to regions of probe arrays, which can then be imaged using charged coupled devices (“CCDs”) for a wide field scanning of the array. Alternatively, another particularly useful method for gathering data from the arrays is through the use of laser confocal microscopy which combines the ease and speed of a readily automated process with high resolution detection. Particularly preferred scanning devices are generally described in, e.g., U.S. Pat. Nos. 5,143,854; 5,424,186; and 6,185,030, all of which are incorporated by reference.

In general, a probe is a surface-immobilized molecule that is recognized by particular target and is sometimes referred to as a ligand. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides or nucleic acids, oligosaccharides, proteins, and monoclonal antibodies.

A target is a molecule that has an affinity for a given probe and is sometimes referred to as a receptor. Targets may be naturally-occurring or manmade molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides or nucleic acids, peptides, cofactors, lectins, sugars, polysaccharides, cells, cellular membranes, and organelles. Targets are sometimes referred to in the art as anti-probes or anti-ligands. As the term “targets” is used herein, no difference in meaning is intended. A “probe target pair” is formed when two macromolecules have combined through molecular recognition to form a complex.

The probe array is preferably fabricated on an optically transparent substrate, but it does not need to be optically transparent. The substrate may be fabricated of a wide range of material, either biological, nonbiological, organic, inorganic, or a combination of any of these, existing as particles, strands, precipitates, gels, sheets, tubing, spheres, containers, capillaries, pads, slices, films, plates, slides, etc. The substrate may have any convenient shape, such as a disc, square, sphere, circle, etc. The substrate is preferably flat but may take on a variety of alternative surface configurations. For example, the substrate may contain raised or depressed regions on which a sample is located. The substrate and its surface preferably form a rigid support on which the sample can be formed. The substrate and its surface are also chosen to provide appropriate light-absorbing characteristics. For instance, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, polycarbonate, or combinations thereof. Other materials with which the substrate can be composed of will be readily apparent to those skilled in the art upon review of this disclosure.

### EXAMPLE

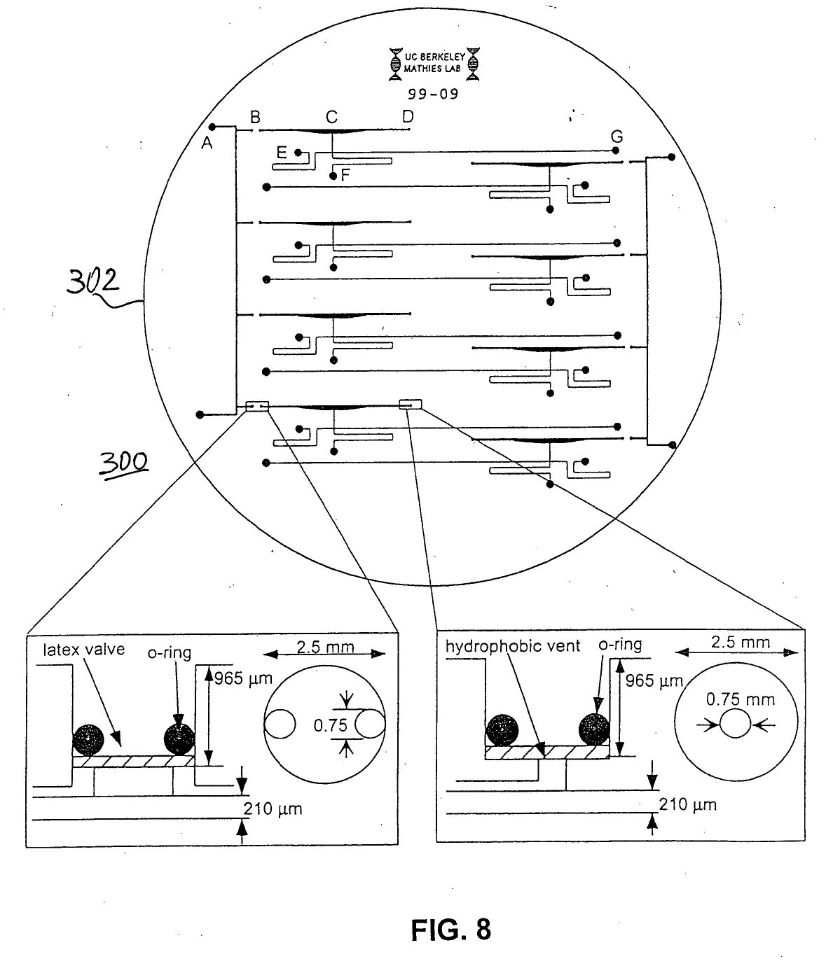

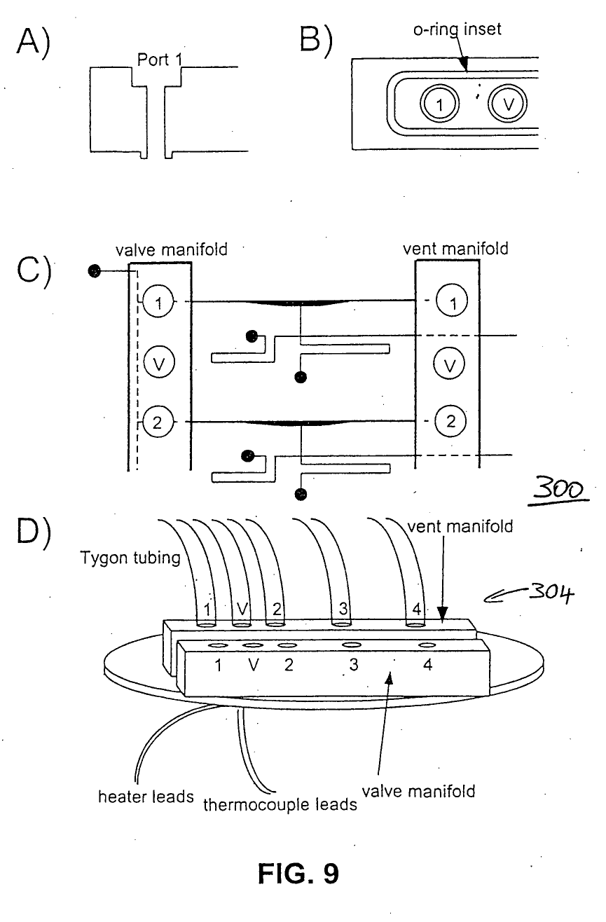

Referring to FIGS. 8 and 9, we developed an integrated DNA amplification and detection device 300 with positive and sensorless microfluidic control to avoid sample losses through manual handling, to minimize the amounts of reagents, and to place reactants positively in the chamber during thermal cycling. This device has also numerous other advantages as the device of Anderson et al. (described by R. C. Anderson, G. J. Bogdan, Z. Barniv, T. D. Dawes, J. Winkler, K. Roy in Microfluidic biochemical analysis system, Proc. 1997 International Conference on Solid-State Sensors and Actuators (Transducers '97), Chicago, USA, Jun. 16-19, 1997, pp. 477-480]. The microfluidic system of Anderson includes valves (and other active components) and hydrophobic vents (and other passive components) in conventionally machined plastic substrates. This system is easily implemented and versatile when operated with volumes in the 1-10 μl range. The device of FIG. 8 extends the Anderson system to submicroliter volume scales.

Integrated device 300 enables the manipulation, amplification, and CE separation of submicroliter volumes of DNA. Device 300 features microfluidic loading and positioning of sample in closed PCR chambers using an active valve and a hydrophobic vent, rapid PCR amplification using thin film heaters, followed by direct injection and rapid separation on a microfabricated CE channel. Device 300 was optimized for temperature uniformity in the reaction chambers (C of FIG. 8) and minimization of chamber volume and total cycle time.

FIG. 8 shows a mask design 302 used to create microfluidic PCR-CE chips. Each valve 304 includes a main chamber with two smaller fluidic ports within it. One port connects to a common fluidic sample bus, while the other connects to a 0.28-μl PCR chamber (chamber C). The PCR chamber is connected additionally to a hydrophobic vent port 306 and to CE separation system 308. The separation system consists of a 5-cm-long separation channel connected to three additional ports, i.e., waste port F, a cathode port E, and anode reservoirs G.

Referring to FIG. 8, integrated device 300 was fabricated using glass wafers (i.e., wafers 1.1-mm thick, 100 mm diameter D263, Schott, Yonkers, N.Y.). Glass wafers were cleaned before deposition of an amorphous silicon sacrificial layer (2000 Å) in a low-pressure chemical vapor deposition (LPCVD) furnace. The wafers were primed with hexamethyldisilazane, spin-coated with photoresist (Shipley 1818, Marlborough, Mass.) at 5000 rpm, and then soft-baked for 30 min at 90° C. A mask pattern shown in FIG. 8 was transferred to the substrate by exposing the photoresist in a Quintel UV contact mask aligner. The photoresist was developed in a 1:1 mixture of Microposit developer concentrate and H₂O. The mask pattern was transferred to the amorphous silicon by a CF₄ plasma etch performed in a plasma-enhanced chemical vapor deposition (PECVD) system (PEII-A, Technics West, San Jose, Calif.). The wafers were etched in a 1:1:2 HF:HCl:H₂O mixture for 7 min at an etch rate of 6 μm/min, giving a final etch depth of 42 μm and a channel width of 100 μm at the bonded surface. The photoresist was stripped and the remaining amorphous silicon removed by a CF₄ plasma etch.

Referring still to FIG. 8, valve and vent structures A and B were formed by drilling a hole to a depth of 965 μm from the back of the etched plate with a 2.5 mm diameter diamond-tipped drill bit (Crystalite, Westerville, Ohio), using a rotary drill press (Cameron, Sonora, Calif.). The depth of these holes was controlled using a micrometer attached to the drill, and horizontal alignment was accomplished using a micrometer translation stage. The valve and vent ports were then drilled through the substrate to the channels using a 0.75 mm diameter diamond-tipped bit. The etched and drilled plate was thermally bonded to a 210-μm-thick flat wafer of identical radius in a programmable vacuum furnace at 560° C. for 3 h (Centurion VPM, J. M. Ney, Yucaipa, Calif.). High quality bonds were typically achieved over the entire substrate. After bonding, the channel surfaces were coated using a modified version of the Hjerten coating protocol [See, e.g., S. M. Clark, R. A. Mathies, Multiplex dsDNA fragment sizing using dimeric intercalation dyes and capillary array electrophoresis; Ionic effects on the stability and electrophoretic mobility of DNA-dye complexes, Anal. Chem. 69 (1997) 1355-1363; or see S. Hjerten, High-performance electrophoresis: elimination of electroendosmosis and solute absorption, J. Chromatogr. 347 (1985) 191-198]. A more detailed discussion of microfabrication methods is presented by P. C. Simpson, A. T. Woolley, R. A. Mathies in Microfabrication technology for the production of capillary array electrophoresis chips, Journal of Biomedical Microdevices 1 (1998) pages 7-26.

A thermal optimization wafer was also constructed using the above-described mask pattern. This wafer was processed as described above, but was etched in 49% HF to a depth of 250 μm, to permit measurement of the actual chamber temperature with a thermocouple probe inserted through the valve structure into the PCR chamber.

Referring to FIGS. 9A, 9B, 9C, and 9D, the valves and vents were controlled by an aluminum manifold depicted in FIG. 9D. Referring to FIG. 9B, each manifold includes an o-ring set into the base of the manifold that seals the manifold to the chip when vacuum is applied to the vacuum seal port (V). The ports each have circular projections that fit into the valve/vent structures and seal against o-rings to hold the valve and vent materials in place. Tygon tubing (⅛-in. OD) connects the manifold system via fluidic connectors (Upchurch, Oak Harbor, Wash.) to a set of computer-controlled solenoid valves that apply vacuum and pressure as required.

Valve and hydrophobic vent materials were installed after fabrication. Latex membranes (i.e., 2.5 mm diameter, thickness approx. 150 μm) were attached to 2.5 mm ID o-rings (made by Apple Rubber Products, Lancaster, N.Y.) with epoxy, and the assembly placed around the projections on the valve manifold. Hydrophobic vent material consisting of circular sections of a 1.0-μm-pore size hydrophobic membrane (Millipore, Bedford, Mass.) was installed similarly.

After wafer fabrication, 1-cm diameter heating elements of resistance 7.8 Ω (Minco #HK5537, Minneapolis, Minn.) and miniature T-type thermocouples (Omega #5TC-TT, Stamford, Conn.) were applied to the back side of the chip with silicone heat sink compound and secured with polyimide tape. The thermocouple was positioned between the heating elements and the chip. For the thermal optimization experiments, a miniature T-type thermocouple (Omega #5TC-TT) was inserted into an enlarged PCR chamber to measure the temperature within the chamber.

Device 300 was optimized by performing several thermal optimization measurements. Specifically, the thermal cycling profile was optimized to ensure accurate heating of the sample. Since the only temperature measured during the PCR amplification was the temperature at the heater, correlations between the measured temperature and the actual chamber temperature were therefore necessary. The 250-μm-deep thermal optimization chip was filled with water and cycled using the thermocouple closest to the heater as the reference thermocouple; the sample thermocouple was placed inside the chamber. Temperature differences between both thermocouples and derivatives of temperature rise were calculated for each temperature step. An adaptive algorithm was used to maximize the rise time derivative and to minimize the temperature difference between thermocouples by adjusting the set parameters of the PID controller after each temperature step.

To optimize the placement of the heating element beneath the PCR chamber, the temperature anisotropy was mapped across the surface of the heater. Using the set-up described above, the thermal optimization chip was filled with water and cycled. The control program captured steady-state temperature data at each of the three temperatures for each cycling run. After each run, the measurement thermocouple was moved horizontally 1.0 mm within the PCR chamber and the run was repeated. The heater was next moved 2.0 mm increments in the lateral direction and the sequence repeated to yield a two-dimensional map of temperature as a function of heater position.

To perform PCR amplification, the PCR chambers were thermally cycled with a Lab-VIEW program (National Instruments, Austin, Tex.). Thermocouple input voltages passed through a signal conditioning unit (National Instruments) and into a 12-bit ADC card (National Instruments) running on a PowerMacintosh 8500 computer. Temperature control was accomplished through a percentage/integrator/differentiator (PID) module within the LabVIEW program. The DAC output used to control the heater passed through a current source circuit to supply the power necessary to drive the heaters.

During heating, the computer turned on the heater until the temperature of the chamber reached the set point; then the PID maintained the temperature to an accuracy of ±0.5° C. When the amplification cycles were completed, the heater was turned off and the chip was allowed to cool passively. The heater was activated again when the temperature reached the set point and completed the timed step. To speed the cooling steps, a computer fan mounted under the microscope stage was activated by the program at the beginning of each cooling step and deactivated at the end of each cooling step. Nitrogen gas was also flowed over the top surface of the chip during the cooling steps. This equipment allowed for very rapid cooling (˜10° C./s), resulting in reduced overall cycling times.

Electrophoretic separations were detected with a laser-excited confocal fluorescence detection system as described previously by A. T. Woolley, R. A. Mathies in Ultra-high-speed DNA fragment separations using microfabricated capillary array electrophoresis chips, Proc. Natl. Acad. Sci. U.S.A. 91 (1994) 11348-11352. Briefly, the chip was placed on a stage and the 488-nm line from an argon ion laser was focused on one of the separation channels at a position 4.6 a 32×(0.4 NA) objective, spatially filtered by a 0.16-mm pinhole, spectrally filtered by a 515-nm bandpass dichroic filter (30-nm band width), and detected by a photomultiplier tube (Products for Research, Danvers, Mass.).

The capillary electrophoresis (CE) separation medium was 0.75% (w/v) hydroxyethyl cellulose (HEC) in 1×Tris borate EDTA (TBE) buffer with 1 μM thiazole orange. The PCR-CE chips were filled with HEC via the vent reservoir (Reservoir C, FIG. 8) by forcing the solution through the entire microfluidic system using a syringe. The gel was evacuated from the PCR chambers and the sample bus by applying vacuum at the valve reservoir, forming a passive barrier to the flow of reagents from the PCR chamber into the separation channel during amplification. The valve and vent manifolds were sealed to the chip by applying vacuum to the port V (FIG. 9C) on each manifold and the sample was introduced at one of the sample bus reservoirs with a pipette. The valve was opened by applying vacuum to the appropriate port on the valve manifold, and vacuum was simultaneously applied to the corresponding hydrophobic vent. Air pressure applied at the sample bus forced the sample through the valve and into the PCR chamber. The valve was then pressure-sealed closed (10-15 psi) to prevent sample movement during heating. Bubble-free loading was consistently achieved using this methodology.

PCR amplification was conducted using a 136 bp amplification product of the M13/pUC19 cloning vector (New England Biolabs, Beverly, Mass.). The 50-μl PCR mixture consisted of Taq MasterMix kit (1×PCR buffer, 1.5 mM MgCl₂, 200 μM each dNTP, and 2.5 U Taq polymerase, Qiagen, Valencia, Calif.), 1.5×10⁻⁵ M BSA, 0.2 μM of each M13/pUC forward and M13/pUC reverse primer (Gibco, Grand Island, N.Y.), and 1×10³ copies of template DNA. The solution was made fresh daily, divided into two 25-μl aliquots, and kept on ice. The on-chip PCR amplification conditions were 20 cycles of 95° C. for 5 s, 53° C. for 15 s, and 72° C. for 10 s, for a total run time of 10 min. Positive controls were run in a conventional Peltier thermal cycler (MJ Research, Watertown, Mass.) at the following conditions: 20 cycles of 95° C. for 1 min, 53° C. for 1 min, and 72° C. for 1 min.

The chip was not moved from valve and vent loading through detection, only the valve manifold was removed from the chip after PCR to provide access for platinum electrodes and for the placement of 1×TBE run buffer in reservoirs D, E, and F (FIG. 8) for injection and separation. After PCR amplification, 112 V/cm was applied for 10 s between reservoirs C and F to inject the M13 PCR product into the separation channel; separation was performed by applying 236 V/cm between reservoirs E and G (FIG. 8). A DNA sizing ladder, pBR322 Mspl (New England Biolabs) was used to verify the size of the PCR product.

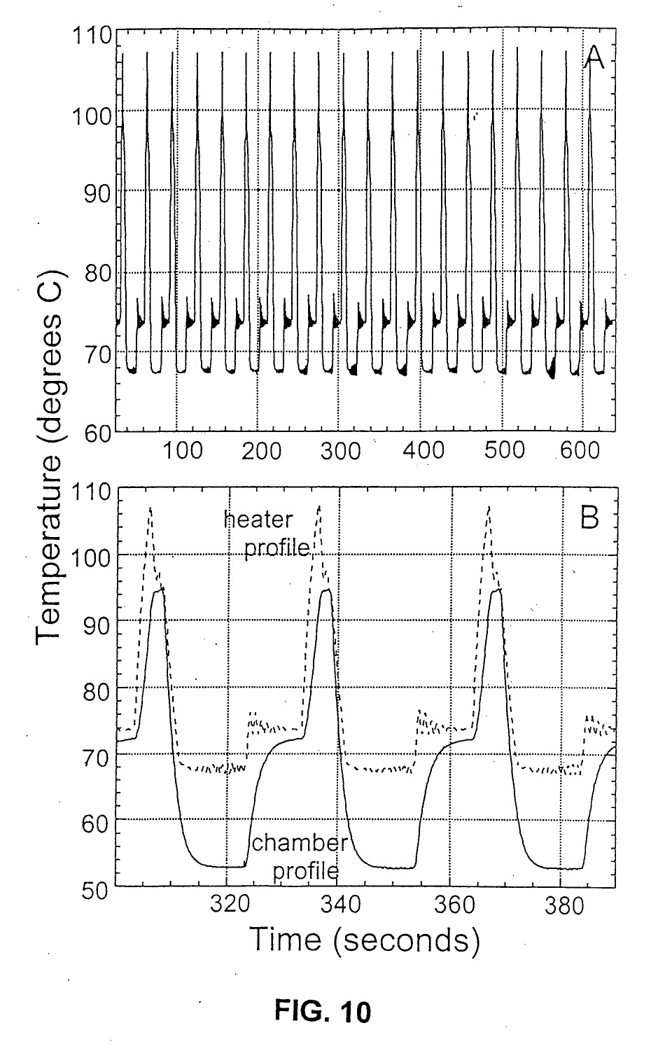

FIGS. 10A and 10B show the temperature profile as a function of time used for the microfluidic PCR-CE amplification and analysis of an M13 amplicon. FIG. 10A shows the entire temperature profile consisting of 20 complete cycles. FIG. 10B shows the thermal cycling profiles of cycles 10, 11, and 12 in detail. The indicated temperature profile is obtained from the standard thermocouple, placed at the heater surface. It was found through consecutive cycling optimization steps using the thermal optimization chip that this temperature profile gave the most accurate and rapid temperature transitions. As a comparison, the temperature readings within the optimization chip using this heating profile are also indicated in FIG. 10B. It was necessary to spike the heater temperature at the beginning of the heating steps to achieve rapid heating within the chamber. The temperature change from the annealing temperature to the extension temperature (72° C.) was made slower than the temperature change from extension to denaturing. Longer times were spent between the annealing temperature and the extension temperature to allow extension while preventing primer melting. Taq polymerase retains some extension capability even at lower temperatures, whereas at higher temperatures primer strands may melt off the template strand, disrupting amplification (as described by H. A. Erlich, in: H. A. Erlich (Ed.), PCR Technology: Principles and Applications for DNA Amplification, Freeman, N.Y., 1989, pp. 1-10). For long extension products, this phenomenon could result in longer extension times, but for short amplification products, even the reduced kinetics of Taq polymerase is sufficient to give complete extension.

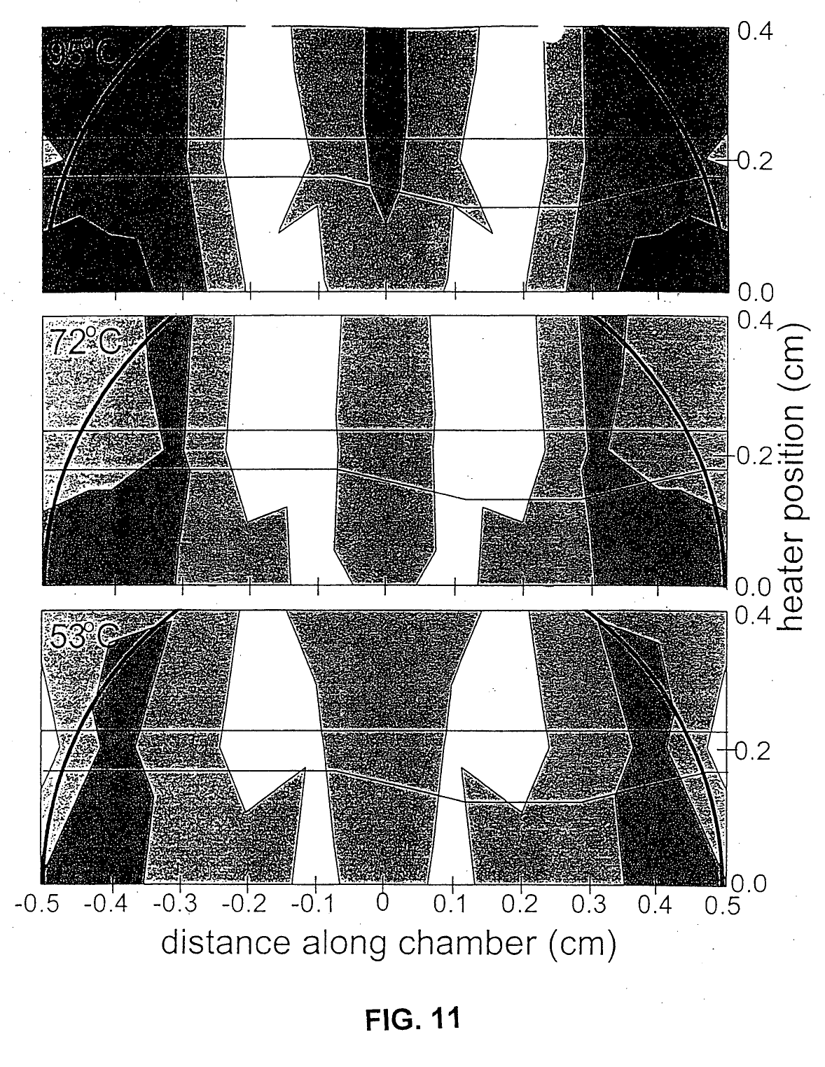

FIGS. 11A, 11B, and 11C are contour plots of the average temperatures over three cycles for each of the three temperatures used in PCR amplification. This temperature was measured for the 250-μm-deep PCR chamber as a function of position across the heater surface on the glass chip. There is notable non-uniformity in the heating across the chamber, especially at the edge. The heater placement used in these experiments was chosen to be 0.2 cm down in the lateral direction and 0.2 cm right in the horizontal direction. The optimum heater placement defined as the location with the minimum temperature anisotropy, is located at approximately 0.05 cm down and centered in the lateral direction. Our heater placement was selected to provide minimal temperature deviation from the set point. The temperatures shown represent an average for each temperature over three amplification cycles. Averaging was done under steady-state conditions by monitoring the final 2 s at 95° C., the final 5 s at 72° C., and the final 10 s at 53° C. Error in these measurements is ±5%, attributed to bubbles and local heating within the optimization chip.

The above temperature analysis assumes that the temperatures obtained using the 250-μm-deep thermal optimization chamber accurately represent the temperatures inside the 42-μm-deep PCR chamber. For the reasons given below, we believe that the results presented here are a good approximation to the actual temperature within the chamber. The thermal conductivity of D263 glass at room temperature is 1.07 W/mK, which is almost three times smaller than that of the water within the chamber. Small thermal conductivities result in larger thermal resistances, which decrease heat transfer. Because the bottom glass plate has the smallest thermal conductivity of any material in the device, it is thus the rate-limiting thermal element in the chip. For this reason, the bottom plate used to form the channels in these experiments was chosen to be as thin as possible (210 μm). The top surface of the chip, although thinner in the thermal optimization chip because of the deeper channels etched into it, maintains a characteristic thickness much larger than that of either the bottom plate or the water within the chamber and will not be greatly affected by a change in channel depth. Thus, differences in the top plate thickness can be neglected in a consideration of heat transfer to the sample.

Differences between the thermal optimization chip and the actual cycling device will result in differences in temperature stabilization time; however, these changes are small compared to the length of the cycling profile. According to a conductive heat transfer theory, the time required for temperature to reach equilibrium in a stationary material is proportional to L²/α, where L is the characteristic thickness of a given region and α is the thermal diffusivity. For a change in depth from 42 to 240 μm, the calculated time required to stabilize at temperature (95° C. is chosen here to demonstrate the largest change possible) increases from 0.0016 to 0.04 s. Since residence times are two orders of magnitude longer than this, any effect is negligible. The last possible effect of temperature measurement in the thermal optimization chip is heat absorption by the thermocouple within the chamber. It is possible that the chamber temperatures within the cycling chip are actually higher than those reported, since during cycling there is no chamber thermocouple present. The exact level of heat absorption by thermocouples in the chamber is not known. Order of magnitude calculations assuming conductive heat transfer to the thermocouple leads indicate that 5% of the applied heat is transferred to the leads. Temperature differences between the cycling chip and the optimization chip at 95° C. averaged 0.6° C., indicating that the optimized PID parameters allowed an effective response to differences between the chips. Additionally, much larger drops in product yield were observed when cycling without the optimized profile, so it is assumed that temperatures measured in the thermal optimization chip are within tolerable error of the actual temperatures within the microfluidic PCR-CE devices.

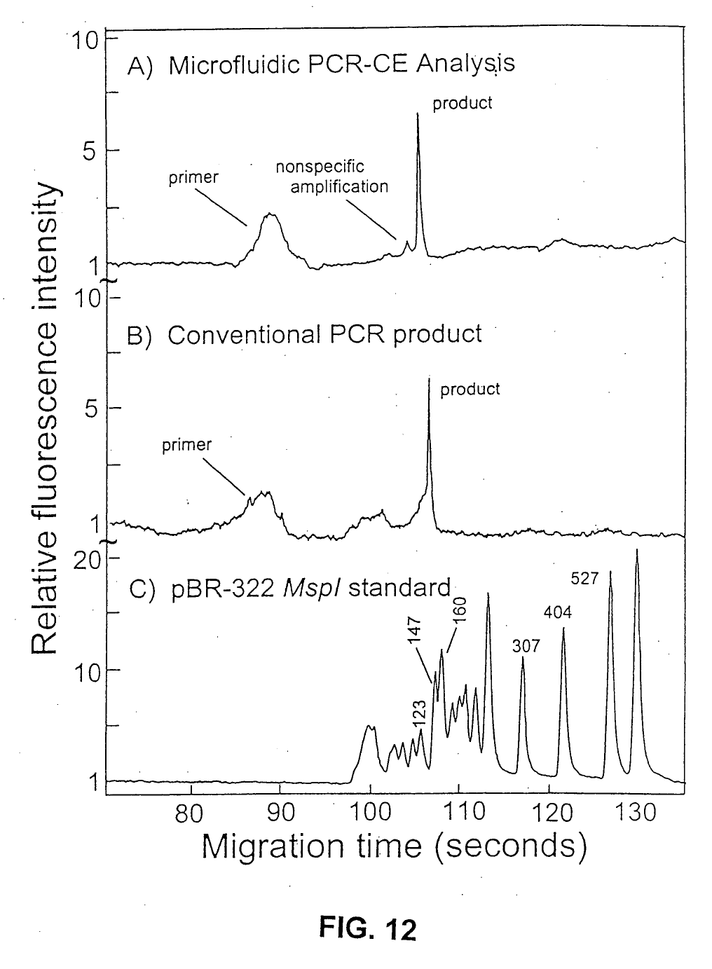

FIG. 12A is a plot of the fluorescent results of an analysis of M13 amplicons conducted on the microfluidic PCR-CE chip. The time for performing 20 cycles of amplification is 10 min. After thermal cycling, the PCR product was immediately injected and separated on the electrophoresis channel. No manual transfer of sample was required, and the entire analysis was complete in less than 15 min. The template concentration used in this amplification was 20 copies/μl, resulting in an average of five to six DNA copies in the chamber before amplification. An examination of the signal-to-noise indicates that 20 cycles of amplification yields a S/N ratio of approximately 7:1. Extrapolation to a S/N ration of 3:1 at this cycle number indicates amplification from only two starting copies in the chamber would be detectable. The use of higher cycle numbers (25-30) to increase the PCR gain would assure detectable signal from a single starting copy in the chamber. Such sensitivity brings this system to the theoretical maximum in sensitivity for PCR amplification. FIG. 12B represents a positive control using the same solution amplified on a Peltier thermal cylinder as for the PCR amplification measured in FIG. 12A. FIG. 12C represents pBR322 Mspl DNA ladder (15 ng/μl) for size comparison.

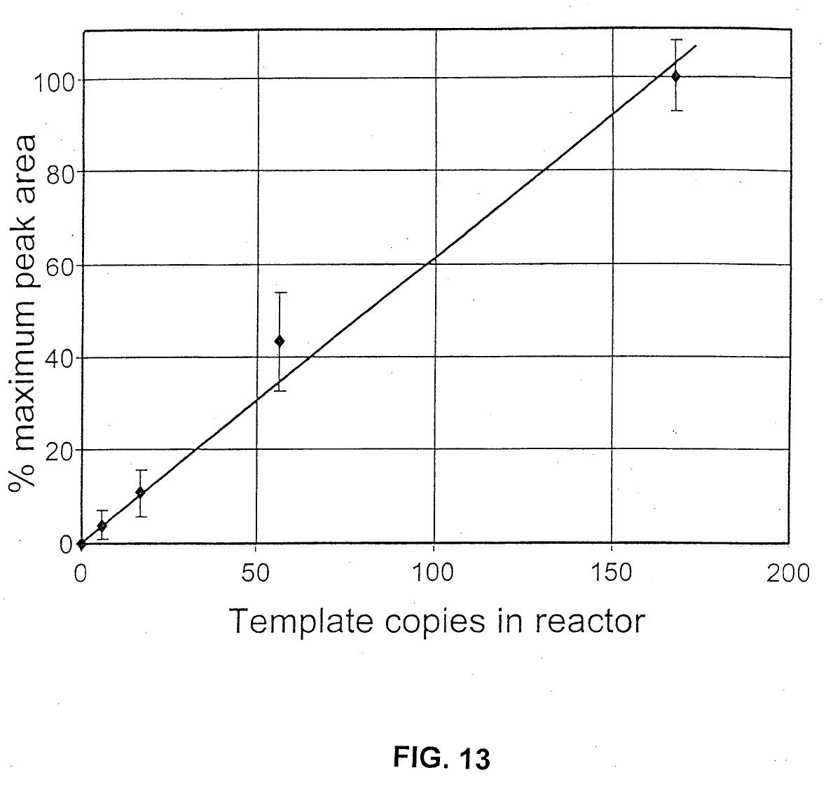

FIG. 13 is a plot of electropherogram product peak area as a function of starting template concentration. As expected, due to the relatively low cycle number and low starting template concentrations, product yield is a linear function of starting template concentration (See e.g., S. Schnell, C. Mendoza, Theoretical description of the polymerase chain reaction, J. Theor. Biol. 188 (1997) 313-318). FIG. 13 demonstrates the expected operation in a regime where reagent-limited losses are insignificant, representing the most powerful amplification possible using this system. Amplification at higher cycle numbers will increase the signal-to-noise, allowing reduction of starting template concentration to single-copy levels, but may decrease linearity. The linearity shown here provides a method for predicting starting template concentration as a monotonic function of product yield, making possible quantitative studies of trace target sequences potentially to the single molecule level.

The microfluidic system used for loading and containing the PCR reaction is critical to its success. Previous attempts to conduct PCR using open sample wells or manual loading of the reactor were unsuccessful. Manual loading often resulted in bubbles within the chamber, and open sample wells led to evaporation and sample movement. The major advantage of the present microfluidic system, aside from its ability to transfer small volumes, is that air bubbles are not introduced into the system. The large temperature changes inherent to PCR drive bubble expansion and contraction; any bubble in the chamber will drive sample movement and cause localized heating. The hydrophobic vent design provides a means for positive, sensorless positioning of the sample during the loading phase. The vent positions the sample and degasses the reaction. The membrane degasses the sample only through diffusion during the PCR reaction, but does provide a slow escape for bubbles should any form during thermal cycling.

The present microfluidic PCR-CE chips demonstrate a number of clear advantages over conventional thermal cycling systems and several improvements over recent small-volume PCR systems. First, the volume cycled (0.28 μl) is the smallest to date, which conserves sample and reduces cost. The dead volume of the microfluidic components used here are 50 nl for each valve and 25 nl for each vent. This is an upper bound, however, as not all ports are filled after the initial sample introduction. Small operating volumes also make the device well-suited to rapid cycling: we have demonstrated 20 cycles of PCR amplification in 10 in. The rate-limiting step is the thermal transition time, rather than energy transfer from the heater to the sample. Another significant advantage gained from the small size of the reactor is the improved molecular limit of detection (LOD) observed here: at a given template concentration, there will be fewer template copies in the smaller reactor. The present device demonstrates an about 100-fold improvement in the LOD compared to other microfabricated thermal cycling devices, and up to 10⁵ improvement over flow-through designs for chemical amplification using continuous-flow PCR on a chip.

The ability to efficiently detect products amplified from low starting concentrations depends in part upon sample injection stacking at the boundary between the PCR chamber and the gel-filled injection channel. The large mobility decrease at this interface results in stacking of the PCR product: as a result, injection times were kept short to avoid overloading the column and fronting effects. One possible limitation of the current design is the non-uniform heating of gel in the injection cross channel. The gel nearest the PCR reactor will be heated more during amplification than the gel nearer the waste reservoir. A small time delay between amplification and injection or the use of smaller or microfabricated heaters more accurately sized to the PCR chamber should reduce or eliminate these temperature-related effects.

The present integrated device eliminates sample handling after the initial loading of the sample bus, which increases assay speed and reproducibility and reduces the possibility of sample contamination from external sources. The entire device is made from inexpensive materials using conventional microfabrication and machining procedures. This reduces the cost of the device, allows for expanded feature density, and also allows the construction of parallel arrays of individually controlled microreactor systems. Further improvements can be obtained by fabricating one or several thin film heaters directly on the chip surface with integrated temperature detection. This improved design reduces the thermal load, resulting in even faster amplification and improved temperature uniformity across the chamber. Extended applications could include performing multiple PCR reactions and multiplex PCR reactions in parallel on a single device, each using separate thermal cycling profiles, and the performance of thermal cycling-based DNA sequencing. The combination of this PCR-CE technology with current sequencing, forensic and medical assays will create powerful new high-throughput methods for DNA amplification and analysis.

Referring again to FIGS. 8 through 9D, the fabricated fully integrated microfluidic device for loading, PCR amplification, and separation of submicroliter volumes of DNA has numerous advantages. This device enables positive and controlled microfluidic sample manipulation, coupled to high-speed, high-sensitivity PCR amplification in a completely enclosed and monolithic chamber, directly linked to high-performance microfabricated capillary electrophoretic separation. The sample volume within the PCR chamber of about 280 nl is very small and the resulting sensitivity is very high for a microfabricated PCR reactor.

While the present invention has been described with reference to the above embodiments and enclosed drawings, the invention is by no means limited to these embodiments and/or embodiments described in the above-cited references (all of which are incorporated by reference). The present invention also includes any modifications or equivalents within the scope of the following claims.

1. A miniature device comprising:
a body having a reaction chamber disposed therein;
a resistive heater electrically connected to a power source for applying power to said heater to facilitate a reaction in said reaction chamber;
a temperature sensor disposed on a surface of said body for determining a temperature within said reaction chamber; and
an appropriately programmed computer for monitoring said temperature and operating said power source to selectively apply said power to said heater.

2. The miniature device of claim 1, further comprising a second reaction chamber fluidly connected to said reaction chamber.

3. The miniature device of claim 2, wherein said second reaction chamber comprises a microcapillary electrophoresis device.

4. The miniature device of claim 2, wherein said second reaction chamber has an oligonucleotide array disposed therein, said oligonucleotide array including a substrate having a plurality of positionally distinct oligonucleotide probes coupled to a surface of said substrate.

5. The miniature device of claim 1, wherein said body comprises at least first and second planar members, said first planar member having a first surface and a well disposed in said first surface, said second planar member having a second surface, said second surface being mated to said first surface whereby said well forms said cavity.

6. The miniature device of claim 5, wherein said temperature sensor is deposited on said second surface wherein when said second surface is mated with said first surface, said temperature sensor on said second surface is positioned within said cavity whereby a temperature at said temperature sensor is substantially the same as a temperature within said cavity.

7. The device of claim 1, wherein said reaction chamber has a volume of from about 0.001 μ1 to about 10 μl.

8. The device of claim 1, wherein said reaction chamber has a volume of from about 0.01 μl to about 1 μl.

9. The device of claim 1, wherein said reaction chamber has a volume of from about 0.05 μl to about 0.5 μl.

10. The device of claim 1, wherein said temperature sensor comprises a thermocouple having a sensing junction positioned adjacent said cavity, and a reference junction positioned outside of said cavity, said thermocouple being electrically connected to a detector for measuring a voltage across said thermocouple.

11. The device of claim 10, wherein said detector for measuring a voltage across said thermocouple measures a DC voltage.

12. The device of claim 10, wherein said thermocouple comprises a first gold film adjoined to a chromium film as said sensing junction and said chromium film adjoined to a second gold film as said reference junction.

13. The device of claim 1, wherein said resistive heater comprises a chromium film and said electrical connection comprises two gold leads overlaying said chromium film and being electrically connected to said power source.

14. The miniature device of claim 1, wherein said first reaction chamber comprises a PCR chamber.

15. The miniature device of claim 1, wherein said vent includes a hydrophobic vent.

16. The miniature device of claim 1 further including a valve constructed to seal input to said reaction chamber.

17. The miniature device of claim 16, wherein said valve includes a diaphragm valve.

18. The miniature device of claim 1, wherein said reaction chamber is in communication with a sealable opening constructed and arranged for introduction of said liquid into said reaction chamber.

19. The miniature device of claim 18, wherein said sealable opening includes a septum.

20. A miniature device comprising:
a body including at least two reaction chambers arranged in parallel, each said reaction chamber being constructed to separately receive a liquid; each said reaction chamber being in fluid communication with a vent;
at least two resistive heaters electrically connected to a power source for applying power; each said heater being constructed to deliver heat to one said reaction chamber;
at least two temperature sensors for determining separately a temperature within said reaction chambers; and
an appropriately programmed computer for monitoring said temperature and operating said power source to selectively apply said power to each said heater to facilitate separate reactions in said reaction chambers,
wherein each said vent enables removal of gas from the corresponding reaction chamber thereby preventing a temperature variation in said liquid during said reaction.

21. The miniature device of claim 20, wherein at least one of said first reaction chambers comprises a PCR chamber.

22. The miniature device of claim 20, wherein at least one of said vents includes a hydrophobic vent.

23. The miniature device of claim 20 further including at least one valve constructed to seal input to at least one said reaction chamber.

24. The miniature device of claim 23, wherein said at least one valve includes a diaphragm valve.

25. The miniature device of claim 20, wherein at least one of said reaction chambers is in communication a sealable opening constructed and arranged for introduction of said liquid into said reaction chamber.

26. The miniature device of claim 25, wherein said sealable opening includes a septum.

27. A miniature device comprising:
a body having a reaction chamber disposed therein and constructed to receive a liquid;
a vent in fluid communication with said reaction chamber;
a resistive heater electrically connected to a power source for applying power to said heater;
a temperature sensor disposed on a surface of said body for determining a temperature within said reaction chamber; and
an appropriately programmed computer for monitoring said temperature and operating said power source to selectively apply said power to said heater to facilitate a reaction in said reaction chamber,
wherein said vent enables removal of gas from said reaction chamber thereby preventing a temperature variation in said liquid during said reaction.
